(19) United States
(12) Patent Application Publication
(10) Pub. No.: US 2009/0274695 A1
(43) Pub. Date: Nov. 5, 2009

(54) Inhibition of urokinase-type plasminogen activator (uPA) activity

(76) Inventors: J. Fernando Bazan, Menlo Park, CA (US); Peter Kirk, San Mateo, CA (US)

Correspondence Address: Goodwin Procter LLP;Attn: Patent Administrator, 135 Commonwealth Drive, Menlo Park, CA, 94025-1105, US

(21) Appl. No.: 12/290,013

(22) Filed: Oct. 23, 2008

Related U.S. Application Data
(60) Provisional application No. 61/013,937, filed on Dec. 14, 2007.
(60) Provisional application No. 61/000,625, filed on Oct. 26, 2007.

(51) Int. Cl. A61K 39/395; A61P 35/00; A61P 35/02; A61P 35/04; A61P 19/02; A61P 27/02; G01N 33/566

(52) U.S. Cl. 424/135.1; 435/377; 514/12; 424/133.1; 424/139.1; 424/136.1; 436/501

(57) ABSTRACT

The invention concerns methods for inhibiting the binding of urokinase-type plasminogen activator (uPA) to its receptor uPAR and/or inhibiting uPA biological activity. The invention further concerns methods for inhibiting tumor formation or metastasis, angiogenesis, such as tumor angiogenesis, and screening assays for identifying CYTL1 agonists.

### CROSS REFERENCE TO RELATED APPLICATIONS

This application is a non-provisional application filed under 37 CFR 1.53(b)(1), claiming priority under 35 USC 119(e) to provisional application number 61/013,937, filed Dec. 14, 2007 and provisional application number 61/000,625, filed Oct. 26, 2007, the contents of which are incorporated herein by reference.

### FIELD OF THE INVENTION

The present invention concerns methods for inhibiting the binding of urokinase-type plasminogen activator (uPA) to its receptor uPAR and/or inhibiting uPA biological activity. The present invention further concerns methods for inhibiting tumor formation or metastasis, angiogenesis, such as tumor angiogenesis, and screening assays for identifying CYTL1 agonists.

### BACKGROUND OF THE INVENTION

Urokinase-type plasminogen activator receptor (uPAR) is structurally unlike any known hemopoietic cytokine receptor. It is a glycosylphosphatidylinositol (GPI)-anchored cell-surface protein, expressed by a wide variety of migratory cell types (Pepper et al. (1993) J Cell Biol 122(3), 673-684; Dano et al. (1999) Apmis 107(1), 120-127; Gyetko et al., (1994) J Clin Invest 93(4), 1380-1387. uPAR has two distinct actions: first, it brings the inactive pro-urokinase-type plasminogen activator (pro-uPA) into close proximity to cell-surface proteases which cleave it to generate urokinase-type plasminogen activator (uPA), which remains tethered to the cell surface (Cubellis et al. (1986) J Biol Chem 261(34), 15819-15822), where it initiates a serine protease cascade leading to pericellular proteolysis (Dano et al., (2005) Thrombosis and Haemostasis 93(4), 676-681; Ploug, M. (2003) Current Pharmaceutical Design 9(19), 1499-1528); secondly, it binds to the extracellular matrix component vitronectin (Wei et al. (1994) J Biol Chem 269(51), 32380-32388), which in turn engages cell-surface integrins (Madsen et al. (2007) J Cell Biol 177(5), 927-939). By affecting both adhesion to and degradation of the extracellular matrix, binding of uPA to uPAR plays an important role in cellular migration, as evidenced by the defect in neutrophil recruitment in uPAR-deficient mice (Rijneveld et al. (2002) J Immunol 168(7), 3507-3511; Gyetko et al. (2000) J Immunol 165(3), 1513-1519).

The biological effects of competitive inhibitors of the uPA-uPAR interaction have been investigated in a number of systems. Tumor cells transfected with a proteolytically inactive mutant form of uPA show reduced capacity for tumor metastasis (Crowly et al. (1993) Proc Natl Acad Sci USA 90(11), 5021-5025), uPA-ATF inhibits tube-formation by microvascular endothelial cells (Kroon et al. (1999) Am J Pathol 154(6), 1731-1742) and angiogenesis following retinal injury (L Gat et al. (2003) Gene Ther 10(25), 2098-2103).

CYTL1 (cytokine-like 1) was cloned as part of a large-scale effort to identify and analyze novel secreted proteins (Clark et al. (2003) Genome research 13(10), 2265-2270), and is disclosed in U.S. Application Publication No. 20050037465 as “PRO4425.” It was found to be identical to C17, the product of a transcript highly expressed in the rare CD34+ subset of hematopoietic stem progenitor cells (Liu et al. (2000) Genomics 65(3), 283-292 (2)). Analysis of source tissues of CYTL1-specific cDNAs, proteomic studies (Hermansson et al., (2004) J Biol Chem 279(42), 43514-43521), and large-scale gene expression analyses (Kumar et al. (2001) Osteoarthritis and cartilage/OARS, Osteoarthritis Research Society 9(7), 641-653; Yager et al. (2004) Genomics 84(3), 524-535) indicate abundant CYTL1 expression in cartilage and bone. Coordinately, the gene encoding CYTL1 in human (NM_{—}018659) maps to chromosome 4:5,067,217-5,072,098, an area (4p16-15) rich in genes implicated in bone and cartilage development (Yager et al., supra; Mangion et al. (1999) American Journal of Human Genetics 65(1), 151-157; Polymeropoulos et al. (1996) Genomics 35(1), 1-5; Shiang et al. (1994) Cell 78(2), 335-342). Analysis of amphipathicity suggests the presence of four alpha-helices (Liu et al. (2000) supra). The cytokine-like nature of CYTL1 has been based on the prediction of amphipathic α-helices in the CYTL1 chain, in a pattern reminiscent of the hemopoietic four α-helix bundle cytokines (Bazan, J. F. (1990) Immunology today 11(10), 350-354). Members of this family have a well-conserved core fold in the absence of signficant sequence similarity (Hill et al., (2002) J Mol Biol 322(1), 205-233), and this protein architecture tends to direct their interaction with a clan of specialized transmembrane receptors (Sprang and Bazan, (1993) Current Opinion in Structural Biology 3(6), 815-827).

### SUMMARY OF THE INVENTION

As discussed above, the interaction between uPA and its receptor uPAR plays a critical role in the migration of a variety of cell-types, both by localizing the initiation of a serine protease cascade to the cell membrane and by modulating associations between cell-surface receptors and the extracellular matrix. The present invention is based, at least in part, on the identification of the secreted, cytokine-like protein CYTL1/C17 as an additional ligand for uPAR, which competes with uPA for uPAR binding.

In one aspect, the invention concerns a method of inhibiting the interaction of a urokinase-type plasminogen activator (uPA) and a urokinase-type plasminogen activator receptor (uPAR) comprising contacting a mixture comprising uPA and uPAR with a cytokine-like 1 (CYTL1) polypeptide or an agonist thereof.

In another aspect, the invention concerns a method of inhibiting a urokinase-type plasminogen activator (uPA) biological activity comprising contacting a cell expressing a urokinase-type plasminogen activator receptor (uPAR) and uPA in vivo with an effective amount of a CYTL1 or an agonist thereof.

In yet another aspect, the invention concerns a method for inhibiting tumor formation or tumor metastasis in a mammalian subject comprising administering to said subject an effective amount of CYTL1 or an agonist thereof.

In a further aspect, the invention concerns a method for inhibiting angiogenesis in a mammalian subject comprising administering to said subject an effective amount of CYTL1 or an agonist thereof.

In a still further aspect, the invention concerns a method of screening for an antagonist of urokinase-type plasminogen activator (uPA), comprising: (a) incubating a mixture containing a urokinase-type plasminogen activator receptor (uPAR) and a CYTL1 or an agonist thereof with a candidate antagonist, and (b) measuring the ability of the candidate antagonist to competitively inhibit the binding of the uPA or agonist thereof to the uPAR.

In all aspects, the agonist may, for example, be polypeptide, a peptide, a non-peptide small molecule, or an agonist CYTL1 antibody or a fragment thereof.

In all aspects, the polypeptide may, for example, be a CYTL1 variant, such as, for example, a CYTL1 variant having at least about 60%, or 65%, or 70%, or 75%, or 80%, or 85%, or 90%, or 95% or 98%, or 99% amino acid sequence identity with the sequence of a native sequence CYTL1 molecule, such as, for example CYTL1 of SEQ ID NO: 2.

In all aspects, the agonist CYTL1 antibody or antibody fragment is preferably monoclonal, and may be chimeric, humanized, or human.

In all aspect, the antibody fragments include, for example, Fab, Fab′, F(ab′)₂, and Fv fragments; diabodies; linear antibodies; single-chain antibody molecules; and multispecific antibodies formed from antibody fragment(s).

In all aspects, the mammalian subject preferably is a human patient.

In all aspects, the tumor or cancer may, for example, be selected from the group consisting of carcinoma, lymphoma, blastoma, sarcoma, and leukemia, including, without limitation, breast cancer, prostate cancer, colon cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma, kidney cancer, vulval cancer, thyroid cancer, hepatic carcinoma and various types of head and neck cancer.

### BRIEF DESCRIPTION OF THE DRAWINGS

FIG. 1: In vivo expression of CYTL1. CYTL1 mRNA expression was analyzed by in situ hybridization performed on (a-d) whole-mount specimens and (e-p) thin sections. All tissues are from mouse and adult unless stated. In (e-j), upper panels are darkfield images, lower panels are corresponding lightfield images. a) day 12 embryo vertebrae b) day 13 embryo footpad c) day 12 embryo trachea and lung d) day 12 embryo ribs e) tendon sheath (tendon (T), metatarsal (M) f) trachea g) pulmonary artery h) day 18 embryo metatarsals i) Higher magnification of articular surfaces in (h), showing CYTL1 expression in superficial chondrocyte layer, and j) Human trachea. White bars indicate 100□m.

FIG. 2: Expression of CYTL1 during chondrocyte differentiation. ATDC5 prechondrocytes were grown to confluence then treated with insulin and ascorbic acid to stimulate differentiation. At timepoints indicated, RNA was harvested and gene expression assayed by TaqMan. Results shown are normalized to GAPDH, and scaled to maximum value observed. Circles—collagen II, triangles—aggrecan, squares—CYTL1. Results are representative of three independent experiments.

FIG. 3: Downregulation of CYTL1 in CIA. (A) Expression levels of IL-1β and CYTL1 were measured by microarray analysis of RNA extracted from joints of mice at various days after CIA protocol intiation. (B) RNA was extracted from footpads of healthy mice and inflamed footpads of mice with CIA. CYTL1 expression was assayed by qRT-PCR, and normalized to β-actin. Mean and SEM (N=5 control/3 CIA) (C) Chondrocyte differentiation was induced in ATDC5 cells for 11 days. Cells were then treated with 0.1 ng/ml IL-1β (filled bars) or medium alone (empty bars) for 24 h. RNA was harvested and assayed by qRT-PCR and normalized to β-actin. Mean and SEM of normalized data from four independent experiments. ‘Rel. expr.’ is expression relative to control.

FIG. 4: uPAR is a receptor for CYTL1. (A) Mock-transfected COS cells and COS cells transfected with uPAR expression construct were incubated with CYTL1-AP or control, TACI-AP, and bound AP activity detected by dye-deposition (B) human and mouse CYTL1-AP were incubated with untransfected COS cells (open bars), or COS cells transfected with murine (hatched bars) or human (solid bars) uPAR. Bound PA activity was measured by colorimetric enzymatic assay. (C) uPAR-transfected cells were incubated with CYTL1-AP and the indicated concentrations of purified CYTL1. Binding of CYTL1-AP was measured by colorimetric enzymatic assay, and expressed as percentage of binding observed in absence of purified CYTL1, after subtraction of background (untransfected cells).

FIG. 5: Surface Plasmon Resonance analysis of the CYTL1-uPAR interaction. Purified CYTL1 at the indicated concentrations was injected over A) immobilized uPAR, B) control flow-cell. C) Equilibrium-binding analysis—peak uPAR—dependent response is plotted against concentration of CYTL1 injected. Curve shows best fit of Langmuir equation to three independent sets of readings. D) The following were injected over immobilized uPAR: i) CYTL1 (1 μM) ii) CYTL1 (1 μM) plus heparin (160 μg/ml) iii) buffer alone, and iv) heparin (160 μg/ml). Overlayed response curves (after subtraction of control flowcell) of three independent preparations of each condition are shown, with average response at equilibrium indicated.

FIG. 6: CYTL1 and uPA compete to bind uPAR. CYTL1-AP was incubated with uPAR-transfected cells in the presence of the indicated concentrations of A) uPA-ATF, B) pro-uPA or C) DFP-inactivated uPA. Cell-surface-bound CYTL1-AP was measured by enzymatic assay, and expressed as percentage of binding in absence of competitor. Curves shown assume reversible competition for a single binding-site. R² values are 0.998, 0.987 and 0.880. D) Recombinant human uPAR (4776 RU) was immobilized in one flowcell of a BIAcore sensor chip. Three identical injections of CYTL1 (1 μM) were performed, before and after injection of 80 μg/ml pro-uPA, and after dissociation of pro-uPA (wash). Equilibrium response after subtraction of control flowcell (BSA immobilized, 5157 RU) is indicated for each CYTL1 injection.

FIGS. 7A and B: Nucleotide sequence (SEQ ID NO: 1) of a native sequence human PRO4425 (CYTL1) cDNA and the deduced amino acid sequence of a native human PRO4425 (CYTL1) polypeptide (SEQ ID NO: 2).

FIG. 8: Enzymatically biotinylated uPAR immobilized on avidin-coated plate probed with CYTL1-AP, binding detected with AP substrate. CYTL1-AP binds directly to uPAR in the absence of other proteins.

FIG. 9: Enzymatically biotinylated uPAR immobilized on avidin-coated plate probed with CYTL1-AP, binding detected with AP substrate. Binding of CYLT1-AP to uPAR is inhibited by anti-iPAR polyclonals.

FIG. 10: BIAcore assay with CYTL1-AP fusion protein.

FIG. 11: CYTL1 inhibits matrigel invasion by PC-3 cells.

FIG. 12: CYTL1 inhibits u-PA dependent cell proliferation.

FIG. 13: Reduced Collagen II expression os observed in chrondrocytes grown in the presence of CYTL1.

FIG. 14: CYTL1 KO mice develop grossly normal cartilage and bone.

FIG. 15: CYTL1 KO mice develop normal bone mineral density.

FIG. 16: CYTL1 KO mice appear to have less severe arthritis.

FIG. 17: Purification scheme for recombinant human CYTL1.

FIGS. 18-21: Screening buffers to optimize stability of CYTL1.

FIG. 22: Purification scheme for purifying mouse CYTL1.

FIG. 23: CYTL1 crystal diffraction results.

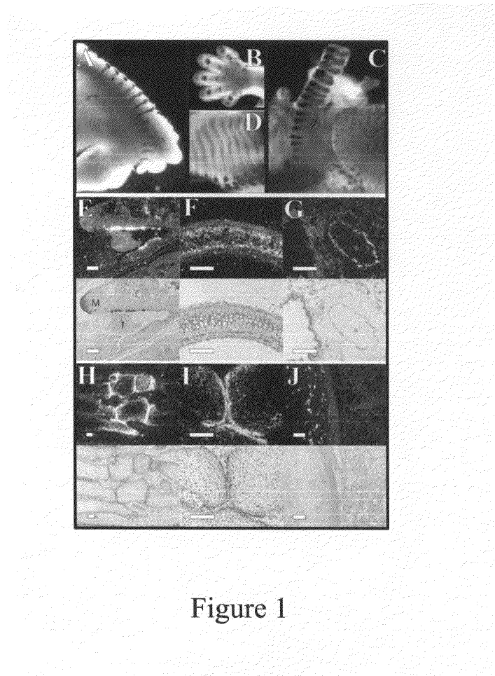

Supplemental FIG. 1: Secondary structure prediction and genomic structure of CYTL1. Alignment of CYTL1 species homologues, with predicted secondary structure below. H: helical, E: extended conformation (β-strand). ‘Jnet Rel’ indicates reliability for the prediction at each residue (9=best). Three helices are readily predicted, along with a short α-helix (only four residues reliably predicted) and β-strand in the AB loop, and a short beta-strand in the C-D loop, characteristic of many short-chain cytokines. Note the proline residues in the C-terminal region, likely to oppose α-helix formation. Positions corresponding to exon boundaries for human CYTL1 are indicated by arrowheads; numbers above the arrowhead indicate the phase of the exon boundary and the intron length. In the four-helical cytokine family, exon boundaries are characteristically found after Helix A, before Helix B, and after Helix C, all in phase zero, as is observed for CYTL1.

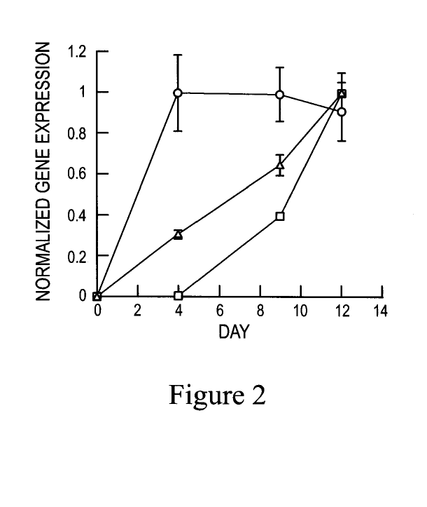

Supplemental FIG. 2: Binding of CYTL1 to ATDC5 cells. A) Mouse CYTL1-AP was used to probe ATDC5 cells at various time-points, as described for transfected COS cells in Materials and Methods. Binding of the fusion protein to cells with morphology of mature chondrocytes was seen from day 4, becoming more widespread at later timepoints. B) Gene expression was assayed by qRT-PCR at various timepoints during ATDC5 maturation. Results shown are normalized to GAPDH, and scaled to maximum value observed. Circles—collagen II, triangles—uPAR, squares—CYTL1.

Table 1: Primers and probes for qRT-PCR. All probes were labeled with FAM reporter dye and TAMRA quencher

### DETAILED DESCRIPTION OF THE PREFERRED EMBODIMENT

1. Definitions

Unless defined otherwise, technical and scientific terms used herein have the same meaning as commonly understood by one of ordinary skill in the art to which this invention belongs. See, e.g., Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present invention, the following terms are defined below.

The term “urokinase-type plasminogen activator” or “u-PA” is used herein to refer to a native-sequence u-PA polypeptide, including the 431 amino acid human prepro-u-PA (NP_{—}002649; Moroi and Aoki, J. Biol. Chem. 251(19), 5956-5965 (1976)) and the corresponding 313-amino acid mature human polypeptide, with or without a 21-amino acid signal sequence (Roldan et al., EMBO J. 1990; 9:467-474), and its native-sequence homologues in a non-human mammal, including all naturally occurring variants, such as alternatively spliced and allelic variants and isoforms, as well as soluble forms thereof.

The terms “CYTL1,” “cytokine-like 1,” and “PRO4425” are used herein interchangeably, and may be isolated from a variety of sources, such as from human tissue types or from another source, or prepared by recombinant or synthetic methods. All disclosures in this specification which refer to a “CYTL1 polypeptide,” or “PRO4425 polypeptide” refer to each of the polypeptides individually as well as jointly. For example, descriptions of the preparation of, purification of, derivation of, formation of antibodies to or against, administration of, compositions containing, treatment of a disease with, etc., pertain to each polypeptide of the invention individually. The term “CYTL1 polypeptide” or “PRO4425 polypeptide” also includes variants of the CYTL1/PRO4425 polypeptides disclosed herein.

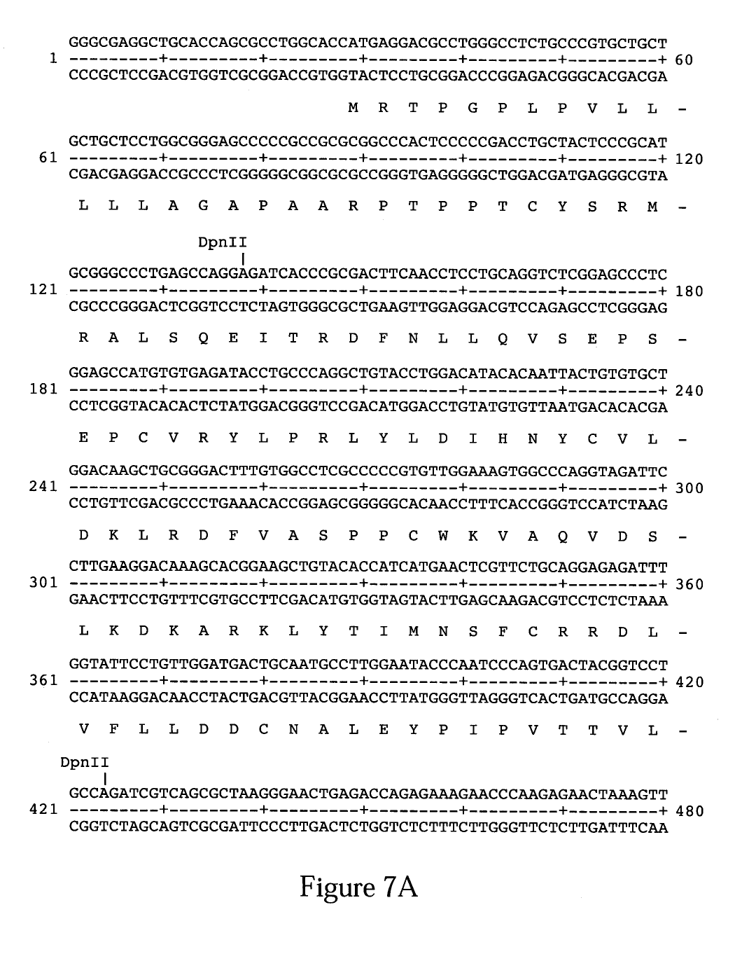

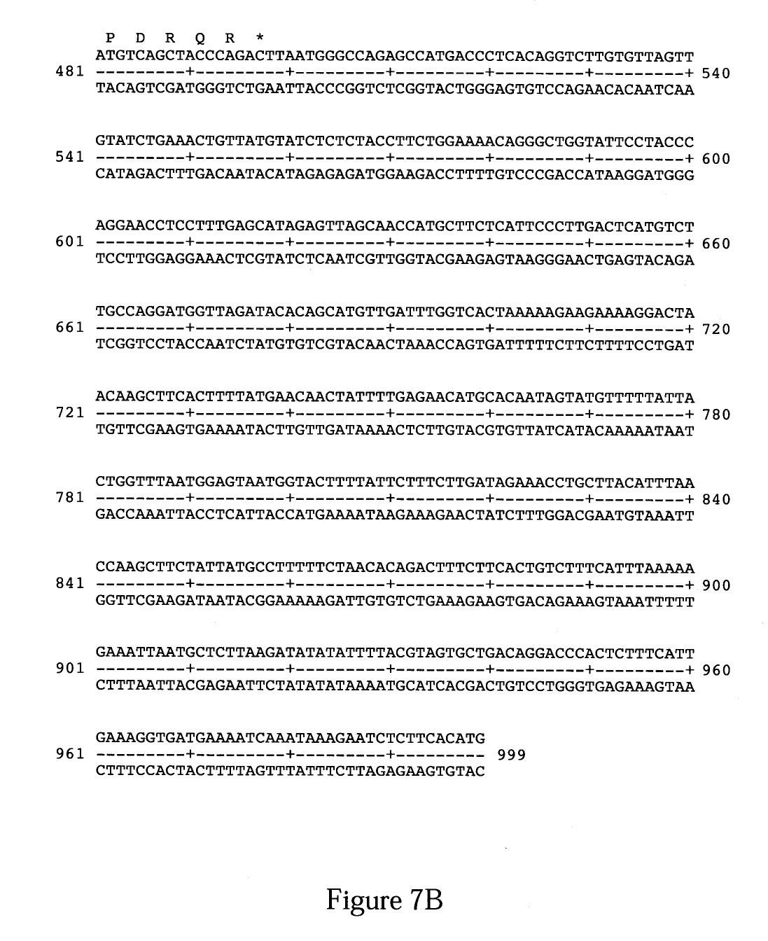

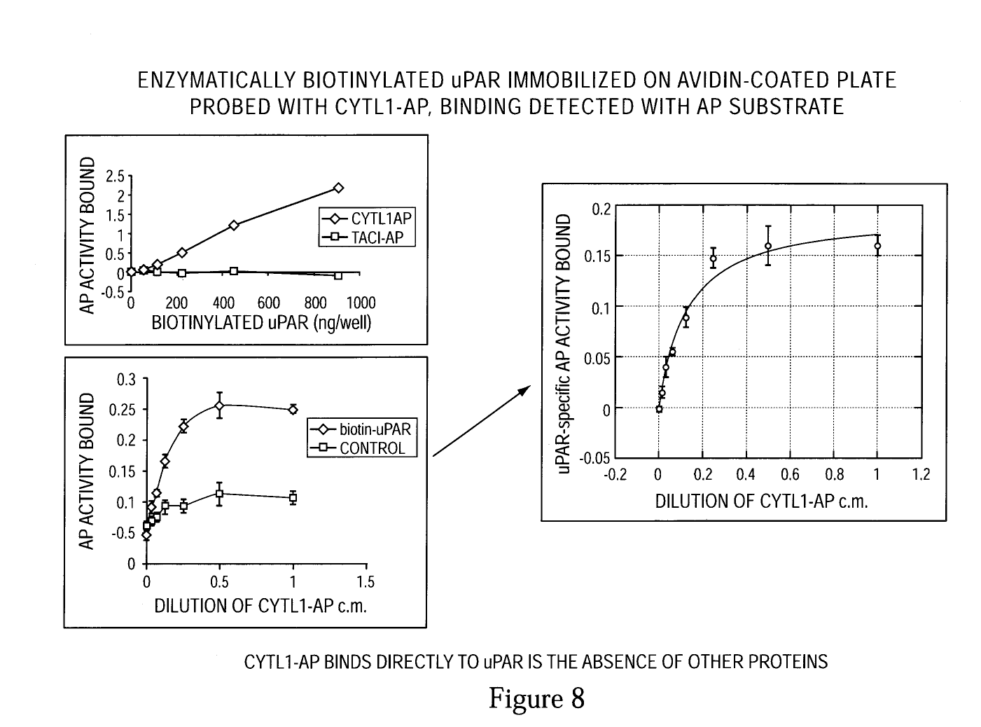

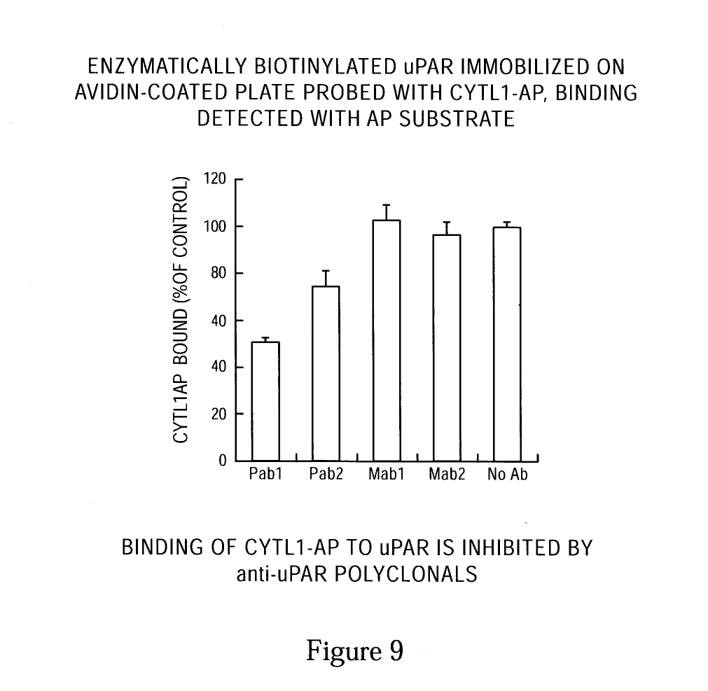

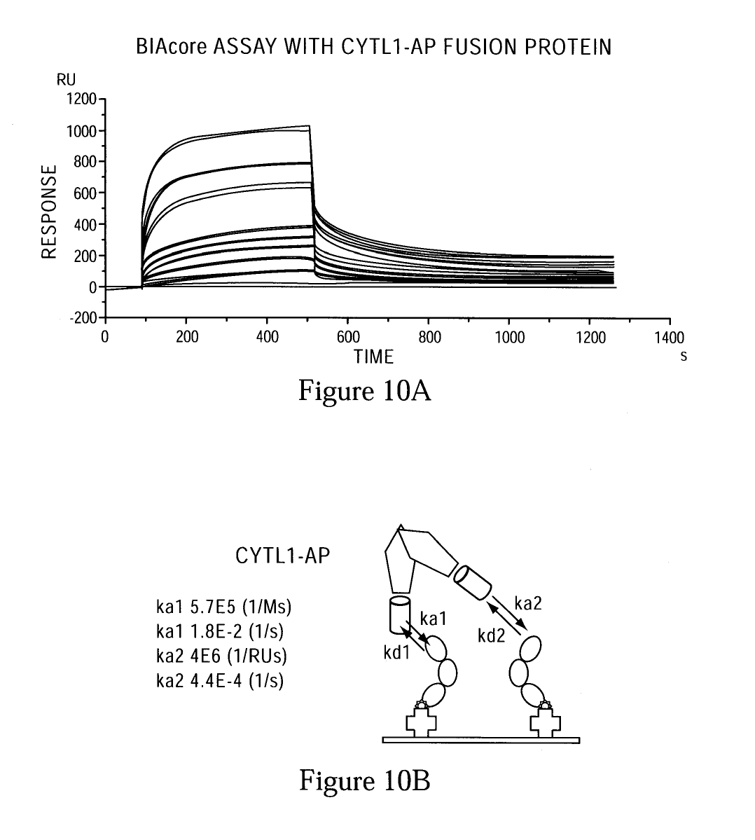

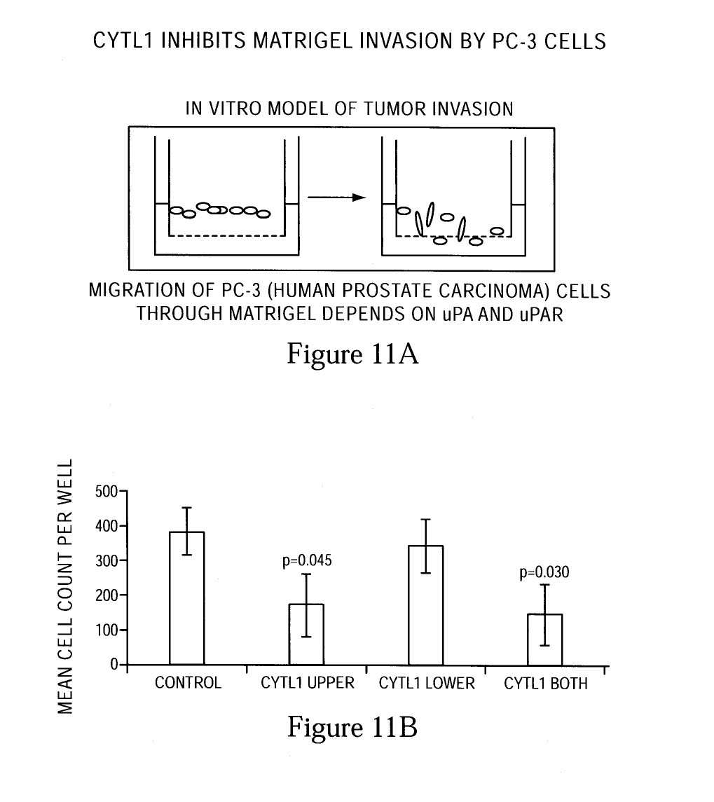

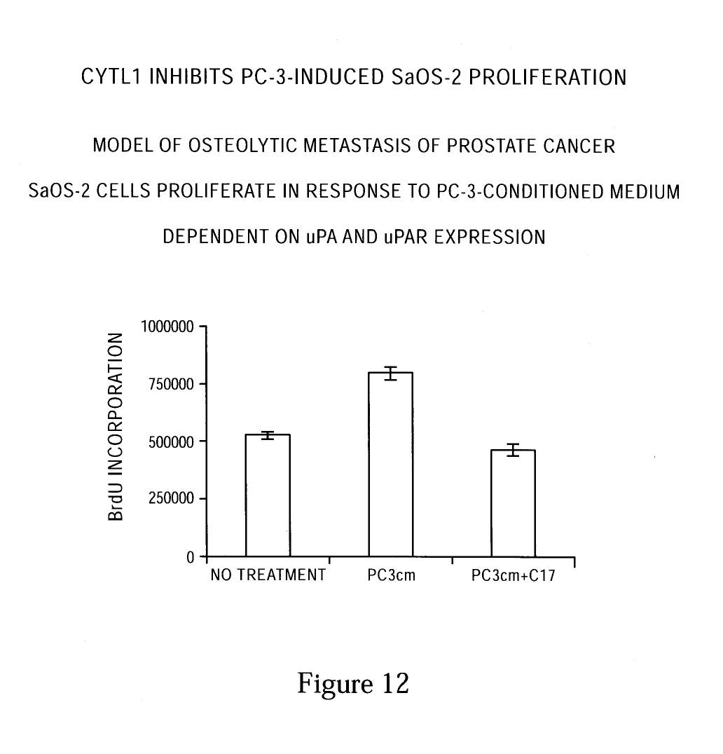

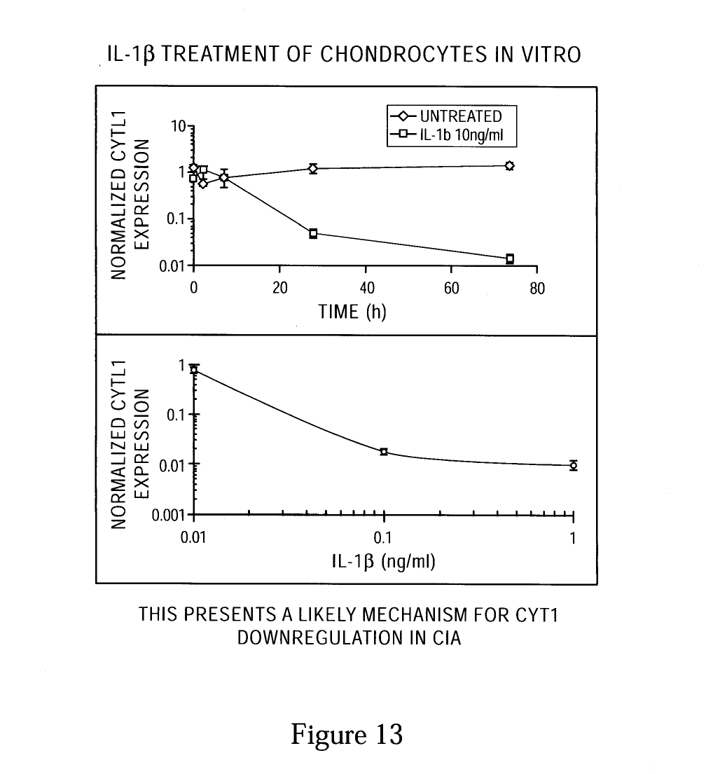

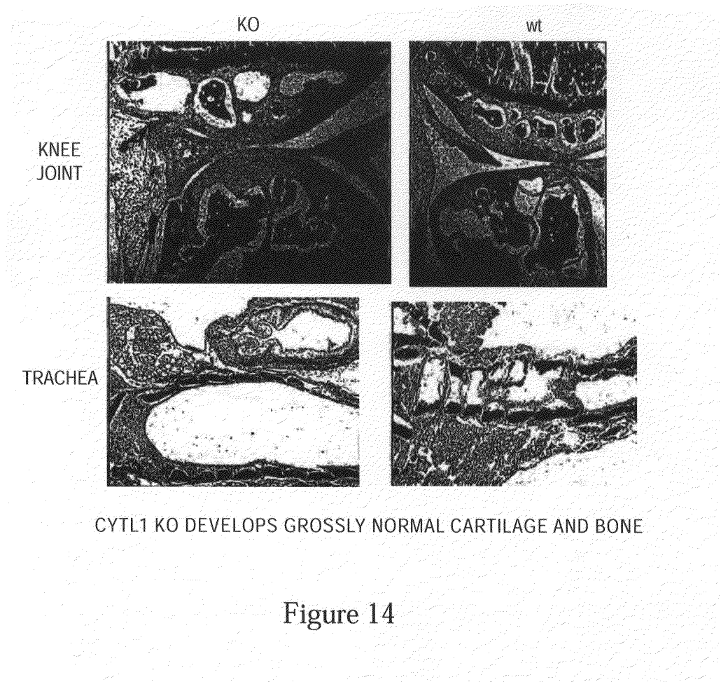

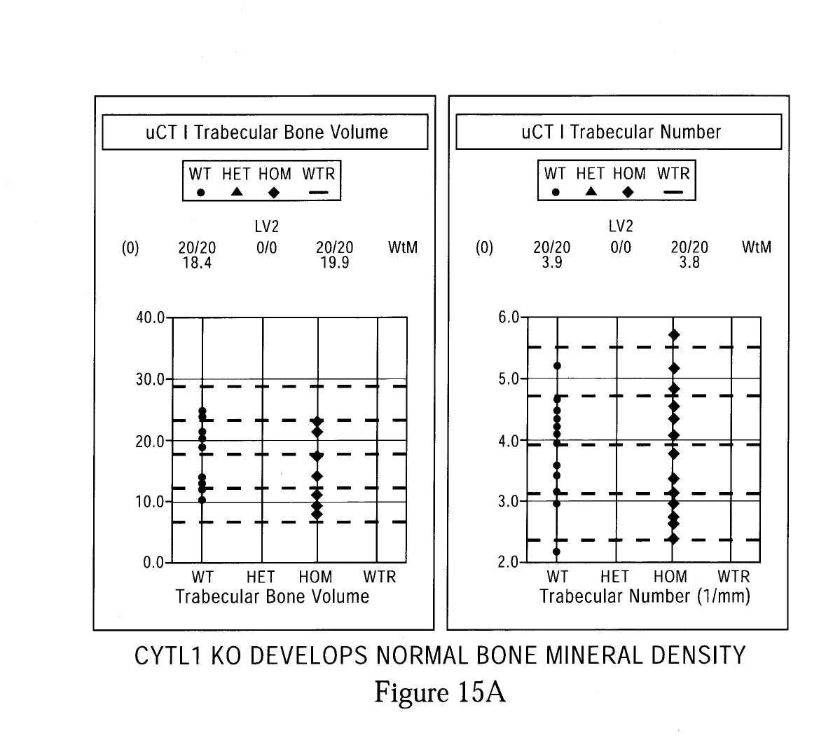

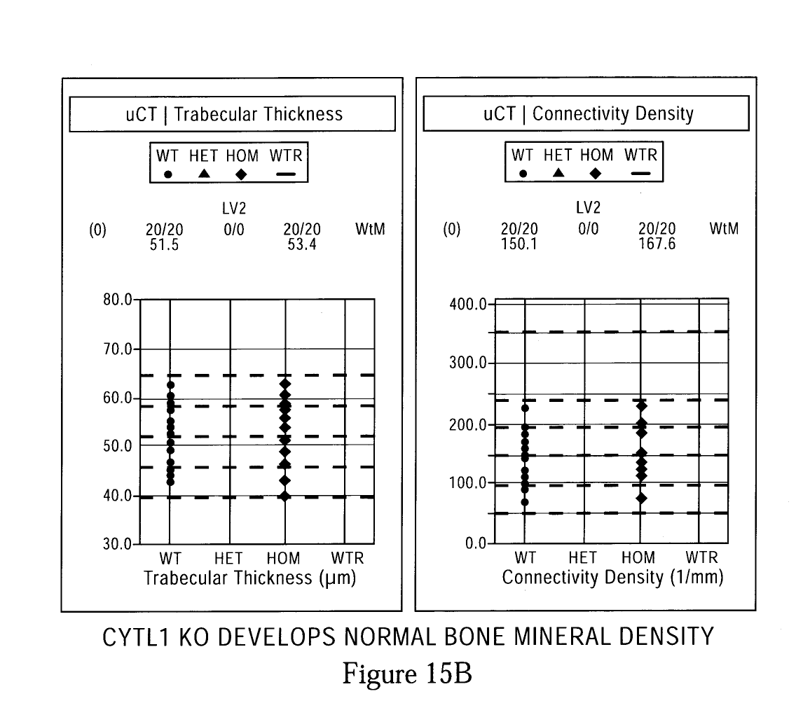

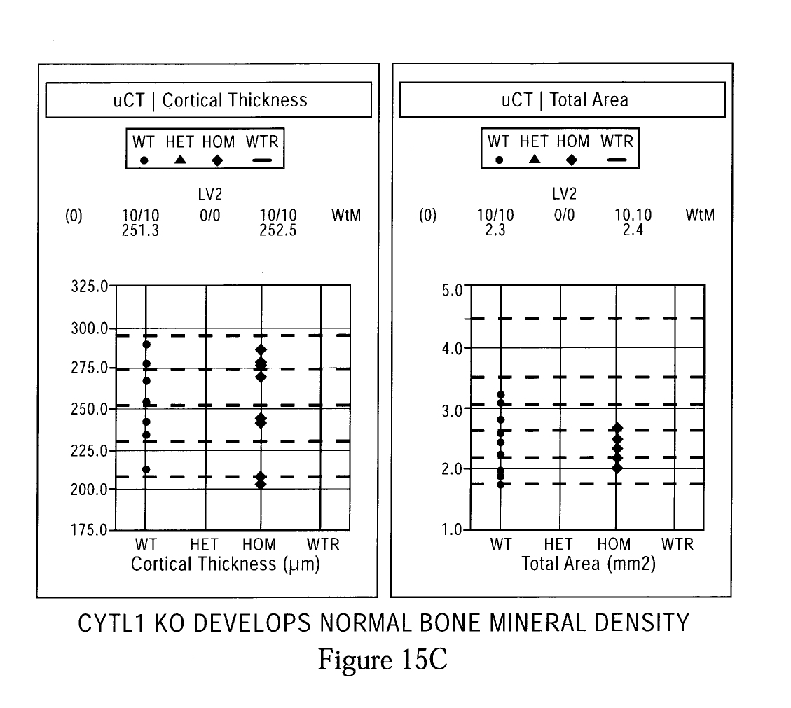

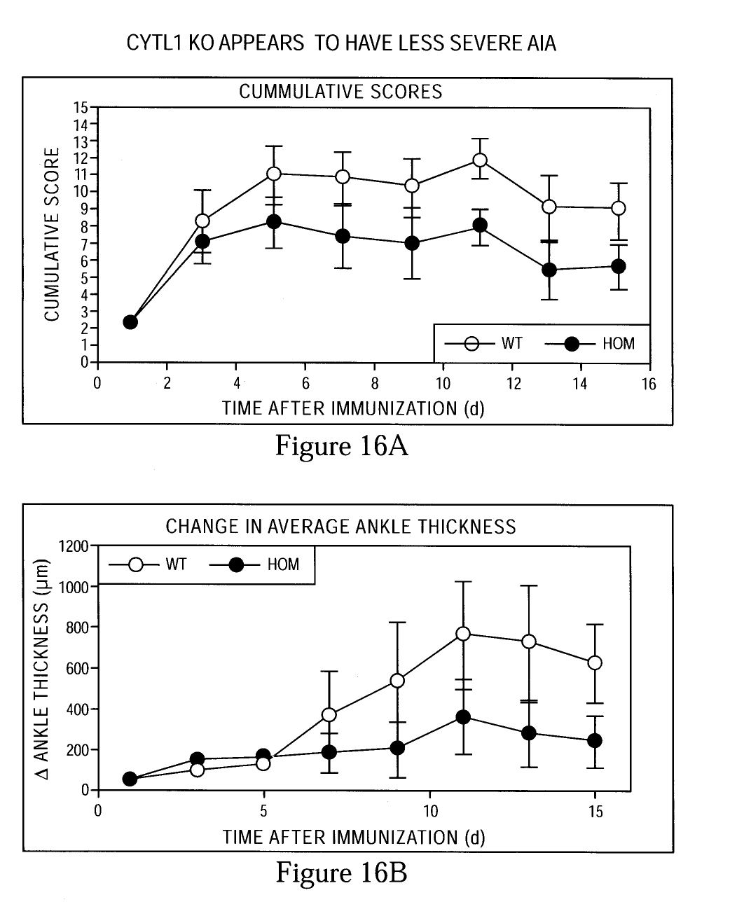

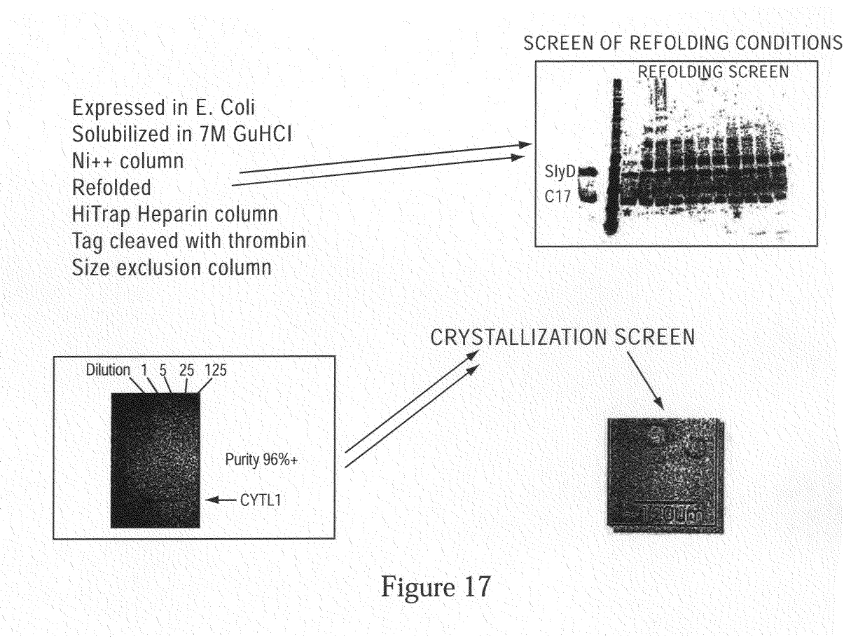

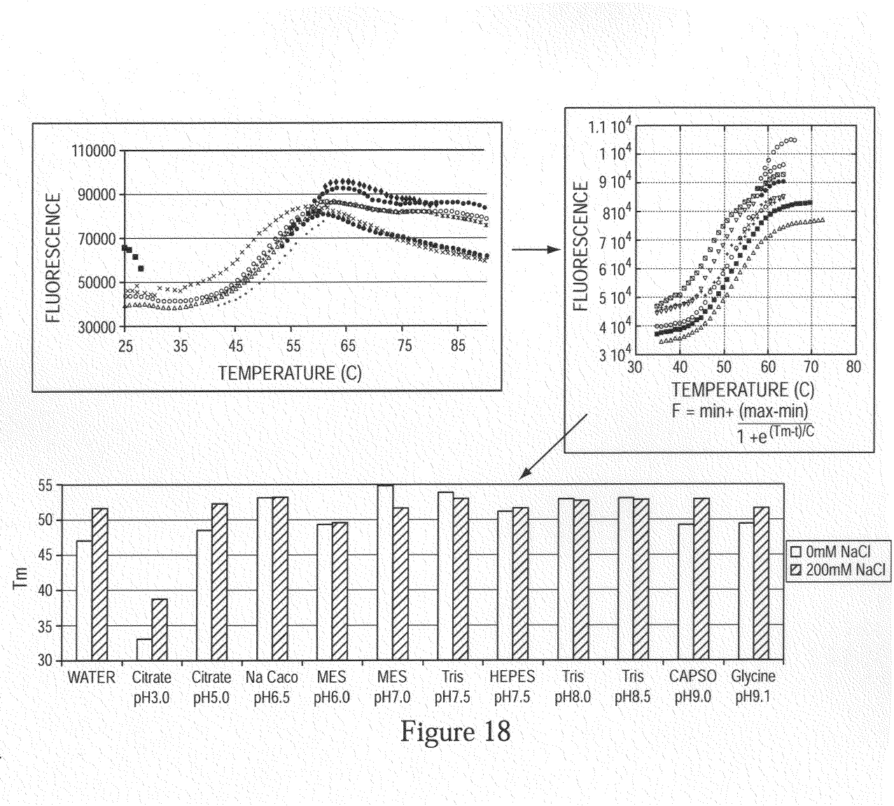

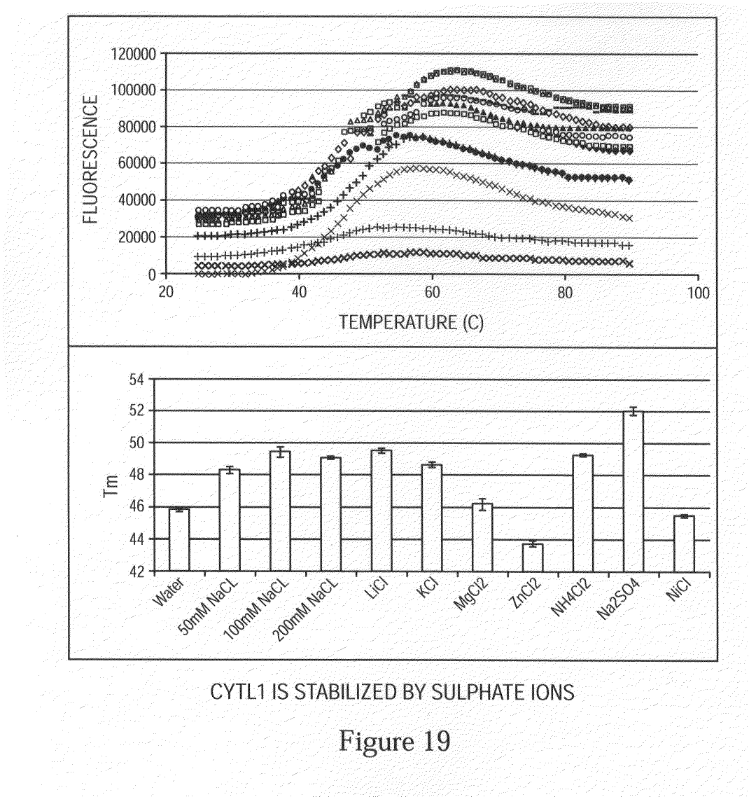

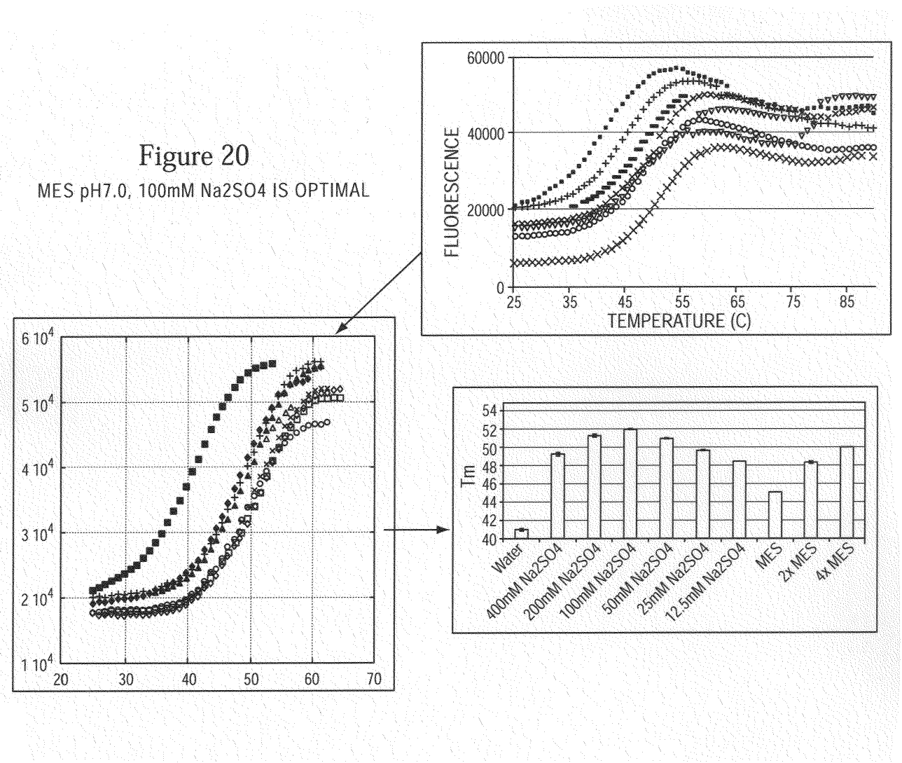

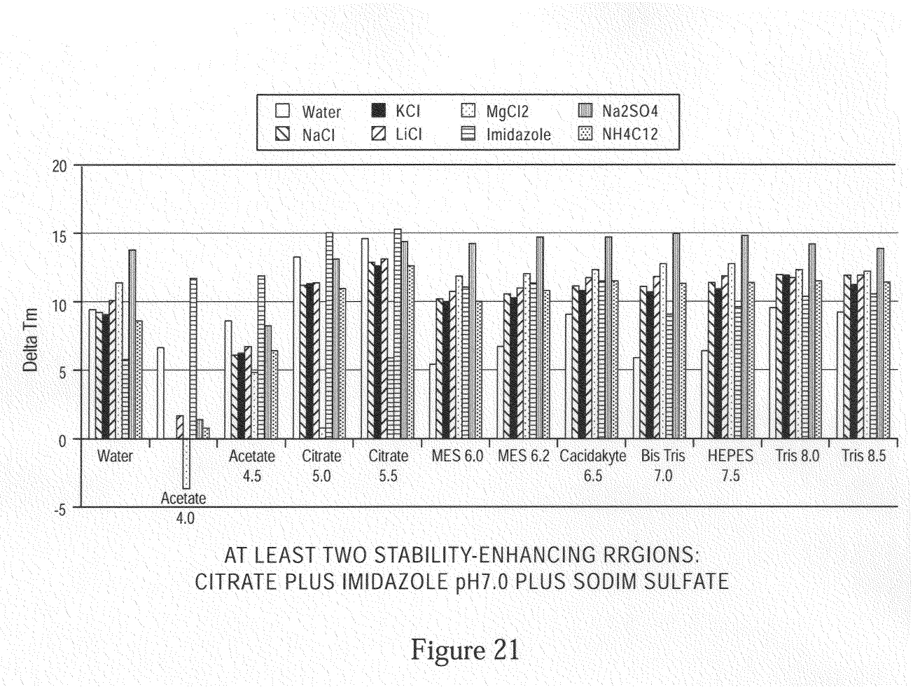

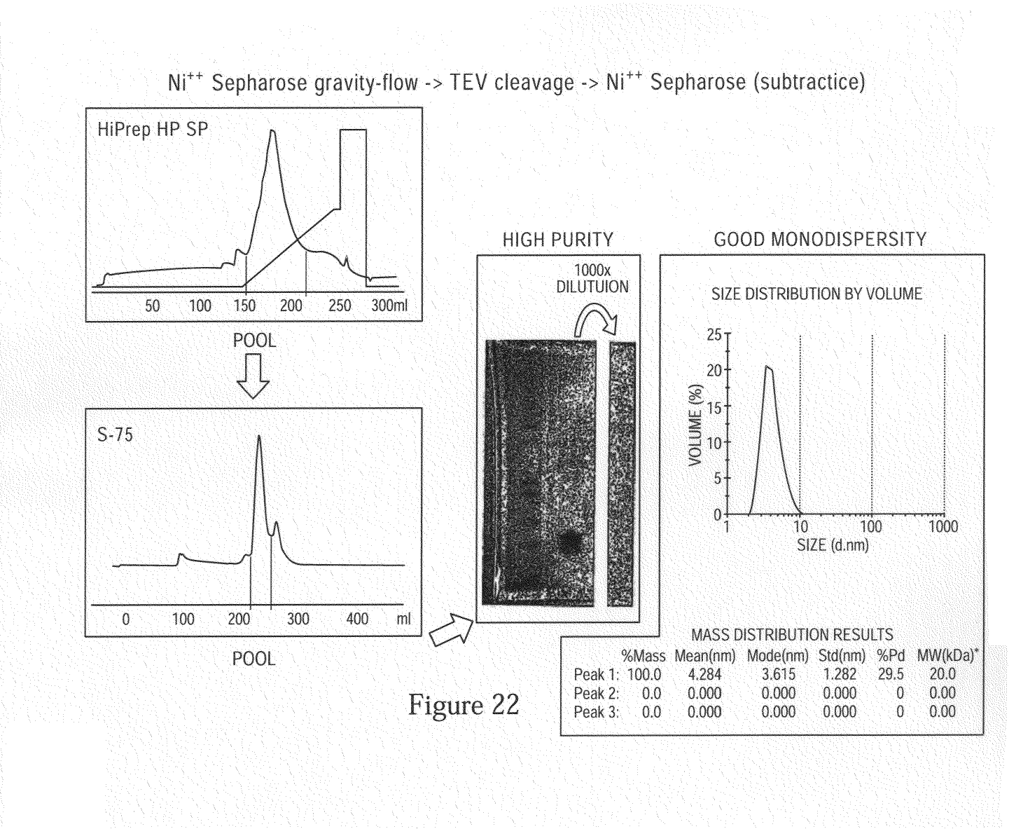

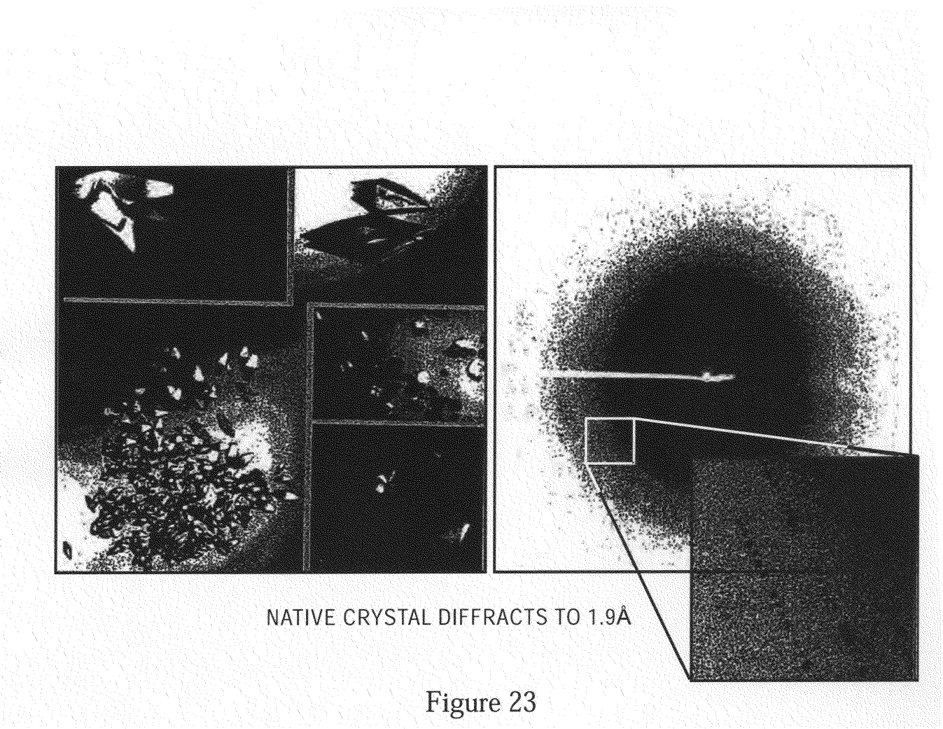

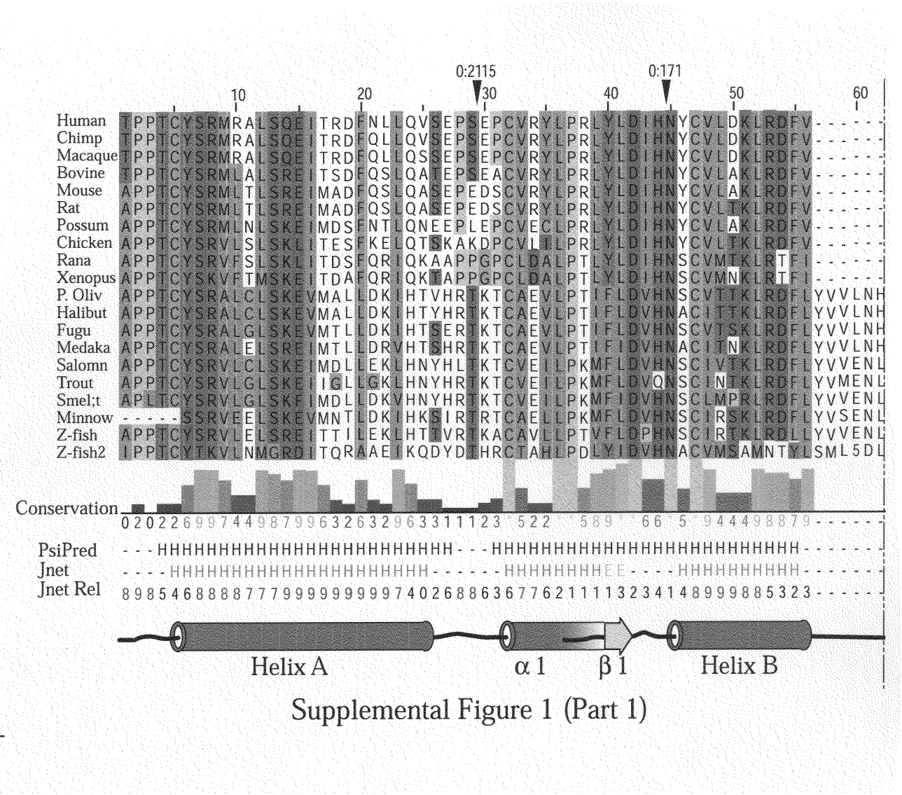

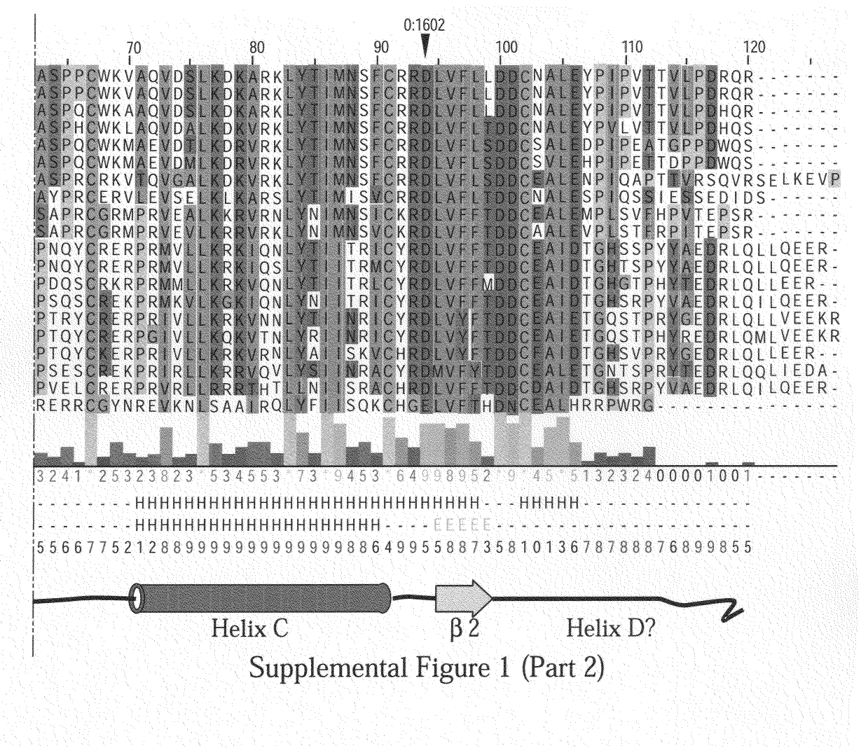

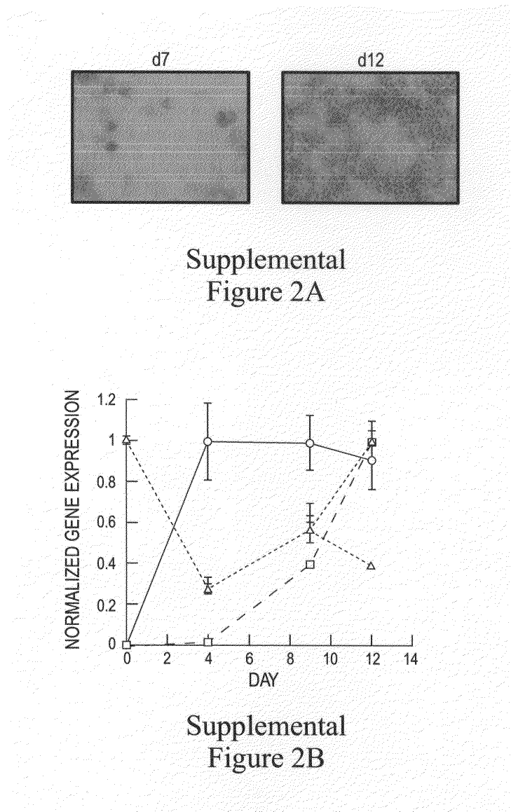

A “native sequence CYTL1/PRO4425 polypeptide” comprises a polypeptide having the same amino acid sequence as the corresponding CYTL1 polypeptide derived from nature. Such native sequence CYTL1 polypeptides can be isolated from nature or can be produced by recombinant or synthetic means. The term “native sequence CYTL1 polypeptide” specifically encompasses naturally-occurring truncated or secreted forms of the specific CYTL1 polypeptide (e.g., an extracellular domain sequence), naturally-occurring variant forms (e.g., alternatively spliced forms) and naturally-occurring allelic variants of the polypeptide. In various embodiments of the invention, the native sequence CYTL1 polypeptide disclosed herein is a mature or full-length native sequence polypeptide comprising the full-length amino acids sequences shown in the accompanying figures. Start and stop codons are shown in bold font and underlined in the figures. However, while the CYTL1 polypeptide disclosed in the accompanying FIG. 7 is shown to begin with a methionine designated by 1, it is conceivable and possible that other methionine residues located either upstream or downstream from the amino acid position 1 in FIG. 7 may be employed as the starting amino acid residue for the CYTL1 polypeptide.

The approximate location of the “signal peptides” of the CYTL1 polypeptide disclosed herein is shown in FIG. 7. It is noted, however, that the C-terminal boundary of a signal peptide may vary, but most likely by no more than about 5 amino acids on either side of the signal peptide C-terminal boundary as initially identified herein, wherein the C-terminal boundary of the signal peptide may be identified pursuant to criteria routinely employed in the art for identifying that type of amino acid sequence element (e.g., Nielsen et al., Prot. Eng. 10:1-6 (1997) and von Heinje et al., Nucl. Acids. Res. 14:4683-4690(1986)). Moreover, it is also recognized that, in some cases, cleavage of a signal sequence from a secreted polypeptide is not entirely uniform, resulting in more than one secreted species. This mature polypeptide, where the signal peptide is cleaved within no more than about 5 amino acids on either side of the C-terminal boundary of the signal peptide as identified herein, and the polynucleotides encoding them, are contemplated by the present invention.

“CYTL1 variant” means an active CYTL1 polypeptide as defined above or below having at least about 80% amino acid sequence identity with a full-length native sequence CYTL1 polypeptide sequence as disclosed herein, a CYTL1 polypeptide sequence lacking the signal peptide as disclosed herein, an extracellular domain of a CYTL1 polypeptide, with or without the signal peptide, as disclosed herein or any other fragment of a full-length CYTL1 polypeptide sequence as disclosed herein. Such CYTL1 polypeptide variants include, for instance, CYTL1 polypeptides wherein one or more amino acid residues are added, or deleted, at the N- or C-terminus of the full-length native amino acid sequence. Ordinarily, a CYTL1 polypeptide variant will have at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to a full-length native sequence CYTL1 polypeptide sequence as disclosed herein, a CYTL1 polypeptide sequence lacking the signal peptide as disclosed herein, an extracellular domain of a CYTL1 polypeptide, with or without the signal peptide, as disclosed herein or any other specifically defined fragment of a full-length CYTL1 polypeptide sequence as disclosed herein. Ordinarily, CYTL1 variant polypeptides are at least about 10 amino acids in length, alternatively at least about 20 amino acids in length, alternatively at least about 30 amino acids in length, alternatively at least about 40 amino acids in length, alternatively at least about 50 amino acids in length, alternatively at least about 60 amino acids in length, alternatively at least about 70 amino acids in length, alternatively at least about 80 amino acids in length, alternatively at least about 90 amino acids in length, alternatively at least about 100 amino acids in length, alternatively at least about 150 amino acids in length, alternatively at least about 200 amino acids in length, alternatively at least about 300 amino acids in length, or more.

“Percent (%) amino acid sequence identity” with respect to the CYTL1 polypeptide sequence identified herein is defined as the percentage of amino acid residues in a candidate sequence that are identical with the amino acid residues in the specific CYTL1 polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, wherein the complete source code for the ALIGN-2 program is provided in Table I below. The ALIGN-2 sequence comparison computer program was authored by Genentech, Inc. and the source code shown in Table I below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table I below. The ALIGN-2 program should be compiled for use on a UNIX operating system, preferably digital UNIX V4.OD. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations using this method, Tables 2 and 3 demonstrate how to calculate the % amino acid sequence identity of the amino acid sequence designated “Comparison Protein” to the amino acid sequence designated “CYTL1,” wherein “CYTL1 ” represents the amino acid sequence of a hypothetical CYTL1 polypeptide of interest, “Comparison Protein” represents the amino acid sequence of a polypeptide against which the “CYTL1 ” polypeptide of interest is being compared, and “X, “Y” and “Z” each represent different hypothetical amino acid residues.

Unless specifically stated otherwise, all % amino acid sequence identity values used herein are obtained as described in the immediately preceding paragraph using the ALIGN-2 computer program. However, % amino acid sequence identity values may also be obtained as described below by using the WU-BLAST-2 computer program (Altschul et al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % amino acid sequence identity value is determined by dividing: (a) the number of matching identical amino acid residues between the amino acid sequence of the CYTL1 polypeptide of interest having a sequence derived from the native CYTL1 polypeptide and the comparison amino acid sequence of interest (i.e., the sequence against which the CYTL1 polypeptide of interest is being compared which may be a CYTL1 variant polypeptide) as determined by WU-BLAST-2 by, and (b) the total number of amino acid residues of the CYTL1 polypeptide of interest. For example, in the statement “a polypeptide comprising an the amino acid sequence A which has or having at least 80% amino acid sequence identity to the amino acid sequence B”, the amino acid sequence A is the comparison amino acid sequence of interest and the amino acid sequence B is the amino acid sequence of the CYTL1 polypeptide of interest.

Percent amino acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the CYTL1 polypeptide natural environment will not be present. Ordinarily, however, isolated polypeptide will be prepared by at least one purification step.

An “isolated” CYTL1 polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions,” as defined herein, may be identified by those that: (1) employ low ionic strength and high temperature for washing, for example 0.015 M sodium chloride/0.0015 M sodium citrate/0.1% sodium dodecyl sulfate at 50.degree. C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×. Denhardt's solution, sonicated salmon sperm DNA (50 .mu.g/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1.times.SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a CYTL1 polypeptide fused to a “tag polypeptide.” The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

“Active” or “activity” for the purposes herein refers to form(s) of a CYTL1 polypeptide which retain a biological and/or an immunological activity of native or naturally-occurring CYTL1, wherein “biological” activity refers to a biological function (either inhibitory or stimulatory) caused by a native or naturally-occurring CYTL1 other than the ability to induce the production of an antibody against an antigenic epitope possessed by a native or naturally-occurring CYTL1 and an “immunological” activity refers to the ability to induce the production of an antibody against an antigenic epitope possessed by a native or naturally-occurring CYTL1. For the purposes of the present invention, preferred biological activities include the ability to bind urokinase-type plasminogen activator receptor (uPAR), to competitively inhibit the binding of uPA to uPAR, and/or to inhibit a uPA biological activity.

The term “uPA biological activity” is used in the broadest sense and includes, without limitation, the ability to bind to uPAR, modulation of cell migration, localization of the initiation of a serine protease cascade to the cell membrane, modulation of associations between cell-surface receptors and the extracellular matrix, participation in the mediation of tumor metastasis, and angiogenic activities.

The term “agonist” is used herein in the broadest sense. A CYTL1 agonist is any molecule that mimics a biological activity mediated by a native sequence CYTL1, regardless of the underlying mechanism. For the purpose of the present invention, the biological activity preferably is the ability to inhibit a uPA biological activity as hereinabove defined. Examples of CYTL1 agonists include, without limitation, agonist anti-CYTL1 antibodies, peptides and non-peptide small organic molecules.

The term “antibody” herein is used in the broadest sense and specifically covers intact antibodies, monoclonal antibodies, polyclonal antibodies, multispecific antibodies (e.g. bispecific antibodies) formed from at least two intact antibodies, and antibody fragments, so long as they exhibit the desired biological activity.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

Antibodies specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include primatized antibodies comprising variable domain antigen-binding sequences derived from a non-human primate (e.g. Old World Monkey, Ape etc) and human constant region sequences.

“Antibody fragments” comprise a portion of an intact antibody, preferably comprising the antigen-binding or variable region thereof. Examples of antibody fragments include Fab, Fab′, F(ab′)₂, and Fv fragments; diabodies; linear antibodies; single-chain antibody molecules; and multispecific antibodies formed from antibody fragment(s).

An “intact” antibody is one which comprises an antigen-binding variable region as well as a light chain constant domain (C_{L}) and heavy chain constant domains, C_{H}1, C_{H}2 and CH3. The constant domains may be native sequence constant domains (e.g. human native sequence constant domains) or amino acid sequence variant thereof. Preferably, the intact antibody has one or more effector functions.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains (Fab, Fab′, F(ab′)₂, Fabc, Fv), in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region” when used herein refers to the regions of an antibody variable domain which are hypervariable in sequence and/or form structurally defined loops. The hypervariable region comprises amino acid residues from a “complementarity determining region” or “CDR” (i.e., residues 24-34, 50-56, and 89-97 in the light chain variable domain and 31-35, 50-65, and 95-102 in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., residues 26-32, 50-52, and 91-96 in the light chain variable domain and 26-32, 53-55, and 96-101 in the heavy chain variable domain; Chothia and Lesk J. Mol. Biol. 196:901-917 (1987)). In both cases, the variable domain residues are numbered according to Kabat et al., supra, as discussed in more detail below. “Framework” or “FR” residues are those variable domain residues other than the residues in the hypervariable regions as herein defined.

A “parent antibody” or “wild-type” antibody is an antibody comprising an amino acid sequence which lacks one or more amino acid sequence alterations compared to an antibody variant as herein disclosed. Thus, the parent antibody generally has at least one hypervariable region which differs in amino acid sequence from the amino acid sequence of the corresponding hypervariable region of an antibody variant as herein disclosed. The parent polypeptide may comprise a native sequence (i.e., a naturally occurring) antibody (including a naturally occurring allelic variant), or an antibody with pre-existing amino acid sequence modifications (such as insertions, deletions and/or other alterations) of a naturally occurring sequence. Throughout the disclosure, “wild type,” “WT,” “wt,” and “parent” or “parental” antibody are used interchangeably.

As used herein, “antibody variant” or “variant antibody” refers to an antibody which has an amino acid sequence which differs from the amino acid sequence of a parent antibody. Preferably, the antibody variant comprises a heavy chain variable domain or a light chain variable domain having an amino acid sequence which is not found in nature. Such variants necessarily have less than 100% sequence identity or similarity with the parent antibody. In a preferred embodiment, the antibody variant will have an amino acid sequence from about 75% to less than 100% amino acid sequence identity or similarity with the amino acid sequence of either the heavy or light chain variable domain of the parent antibody, more preferably from about 80% to less than 100%, more preferably from about 85% to less than 100%, more preferably from about 90% to less than 100%, and most preferably from about 95% to less than 100%. The antibody variant is generally one which comprises one or more amino acid alterations in or adjacent to one or more hypervariable regions thereof.

An “amino acid alteration” refers to a change in the amino acid sequence of a predetermined amino acid sequence. Exemplary alterations include insertions, substitutions and deletions. An “amino acid substitution” refers to the replacement of an existing amino acid residue in a predetermined amino acid sequence; with another different amino acid residue.

A “replacement” amino acid residue refers to an amino acid residue that replaces or substitutes another amino acid residue in an amino acid sequence. The replacement residue may be a naturally occurring or non-naturally occurring amino acid residue.

An “amino acid insertion” refers to the introduction of one or more amino acid residues into a predetermined amino acid sequence. The amino acid insertion may comprise a “peptide insertion” in which case a peptide comprising two or more amino acid residues joined by peptide bond(s) is introduced into the predetermined amino acid sequence. Where the amino acid insertion involves insertion of a peptide, the inserted peptide may be generated by random mutagenesis such that it has an amino acid sequence which does not exist in nature. An amino acid alteration “adjacent a hypervariable region” refers to the introduction or substitution of one or more amino acid residues at the N-terminal and/or C-terminal end of a hypervariable region, such that at least one of the inserted or replacement amino acid residue(s) form a peptide bond with the N-terminal or C-terminal amino acid residue of the hypervariable region in question.

A “naturally occurring amino acid residue” is one encoded by the genetic code, generally selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val).

A “non-naturally occurring amino acid residue” herein is an amino acid residue other than those naturally occurring amino acid residues listed above, which is able to covalently bind adjacent amino acid residues(s) in a polypeptide chain. Examples of non-naturally occurring amino acid residues include norleucine, ornithine, norvaline, homoserine and other amino acid residue analogues such as those described in Ellman et al. Meth. Enzym. 202:301-336 (1991). To generate such non-naturally occurring amino acid residues, the procedures of Noren et al., Science 244:182 (1989) and Ellman et al., supra, can be used. Briefly, these procedures involve chemically activating a suppressor tRNA with a non-naturally occurring amino acid residue followed by in vitro transcription and translation of the RNA.

Throughout this disclosure, reference is made to the numbering system from Kabat, E. A., et al., Sequences of Proteins of Immunological Interest (National Institutes of Health, Bethesda, Md. (1987) and (1991). In these compendiums, Kabat lists many amino acid sequences for antibodies for each subclass, and lists the most commonly occurring amino acid for each residue position in that subclass. Kabat uses a method for assigning a residue number to each amino acid in a listed sequence, and this method for assigning residue numbers has become standard in the field. The Kabat numbering scheme is followed in this description. For purposes of this invention, to assign residue numbers to a candidate antibody amino acid sequence which is not included in the Kabat compendium, one follows the following steps. Generally, the candidate sequence is aligned with any immunoglobulin sequence or any consensus sequence in Kabat. Alignment may be done by hand, or by computer using commonly accepted computer programs; an example of such a program is the Align 2 program. Alignment may be facilitated by using some amino acid residues which are common to most Fab sequences. For example, the light and heavy chains each typically have two cysteines which have the same residue numbers; in V_{L} domain the two cysteines are typically at residue numbers 23 and 88, and in the V_{H} domain the two cysteine residues are typically numbered 22 and 92. Framework residues generally, but not always, have approximately the same number of residues, however the CDRs will vary in size. For example, in the case of a CDR from a candidate sequence which is longer than the CDR in the sequence in Kabat to which it is aligned, typically suffixes are added to the residue number to indicate the insertion of additional residues (see, e.g., residues 100abc in FIG. 1B). For candidate sequences which, for example, align with a Kabat sequence for residues 34 and 36 but have no residue between them to align with residue 35, the number 35 is simply not assigned to a residue.

As used herein, an antibody with a “high-affinity” is an antibody having a K_{D}, or dissociation constant, in the nanomolar (nM) range or better. A K_{D} in the “nanomolar range or better” may be denoted by X nM, where X is a number less than about 10.

The term “filamentous phage” refers to a viral particle capable of displaying a heterogenous polypeptide on its surface, and includes, without limitation, fl, fd, Pfl, and M13. The filamentous phage may contain a selectable marker such as tetracycline (e.g., “fd-tet”). Various filamentous phage display systems are well known to those of skill in the art (see, e.g., Zacher et al., Gene 9: 127-140 (1980), Smith et al., Science 228: 1315-1317 (1985); and Parmley and Smith, Gene 73: 305-318 (1988)).

The term “panning” is used to refer to the multiple rounds of screening process in identification and isolation of phages carrying compounds, such as antibodies, with high affinity and specificity to a target.

The terms “treating,” “treatment” and “therapy” as used herein refer to curative therapy, prophylactic therapy, and preventative therapy. Consecutive treatment or administration refers to treatment on at least a daily basis without interruption in treatment by one or more days. Intermittent treatment or administration, or treatment or administration in an intermittent fashion, refers to treatment that is not consecutive, but rather cyclic in nature.

The term “mammal” as used herein refers to any mammal classified as a mammal, including humans, higher non-human primates, rodents, domestic and farm animals, such as cows, horses, dogs and cats. In a preferred embodiment of the invention, the mammal is a human.

Administration “in combination with” one or more further therapeutic agents includes simultaneous (concurrent) and consecutive administration in any order.

An “effective amount” is an amount sufficient to effect beneficial or desired therapeutic (including preventative) results. An effective amount can be administered in one or more administrations.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. The term “progeny” refers to any and all offspring of every generation subsequent to an originally transformed cell or cell line. Mutant progeny that have the same function or biological activity as screened for in the originally transformed cell are included. Where distinct designations are intended, it will be clear from the context.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include, without limitation, breast cancer, prostate cancer, colon cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma, kidney cancer, vulval cancer, thyroid cancer, hepatic carcinoma and various types of head and neck cancer.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

A “small molecule” is defined herein to have a molecular weight below about 1000 Daltons, preferably below about 500 Daltons.

An “anti-angiogenic agent” refers to a compound which blocks, or interferes with to some degree, the development of blood vessels. The anti-angiogenic factor may, for instance, be a small molecule or antibody that binds to a growth factor or growth factor receptor involved in promoting angiogenesis. The preferred anti-angiogenic factor herein is an antibody that binds to vascular endothelial growth factor (VEGF), such as bevacizumab (AVASTIN®).

The term “anti-neoplastic composition” refers to a composition useful in treating cancer comprising at least one active therapeutic agent, e.g., “anti-cancer agent.” Examples of therapeutic agents (anti-cancer agents) include, but are limited to, e.g., chemotherapeutic agents, growth inhibitory agents, cytotoxic agents, agents used in radiation therapy, anti-angiogenesis agents, apoptotic agents, anti-tubulin agents, and other-agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva™), platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also included in the invention.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

l A “chemotherapeutic agent” is a chemical compound useful in the treatment of cancer. Examples of ®chemotherapeutic agents include alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; TLK 286 (TELCYTA™); acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; bisphosphonates, such as clodronate; antibiotics such as the enediyne antibiotics (e. g., calicheamicin, especially calicheamicin gamma II and calicheamicin omega II (see, e.g., Agnew, Chem Intl. Ed. Engl., 33: 183-186 (1994)) and anthracyclines such as annamycin, AD 32, alcarubicin, daunorubicin, dexrazoxane, DX-52-1, epirubicin, GPX-100, idarubicin, KRN5500, menogaril, dynemicin, including dynemicin A, an esperamicin, neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin, liposomal doxorubicin, and deoxydoxorubicin), esorubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; folic acid analogues such as denopterin, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replenisher such as folinic acid (leucovorin); aceglatone; anti-folate anti-neoplastic agents such as ALIMTA7, LY231514 pemetrexed, dihydrofolate reductase inhibitors such as methotrexate, anti-metabolites such as 5-fluorouracil (5-FU) and its prodrugs such as UFT, S-1 and capecitabine, and thymidylate synthase inhibitors and glycinamide ribonucleotide formyltransferase inhibitors such as raltitrexed (TOMUDEX™, TDX); inhibitors of dihydropyrimidine dehydrogenase such as eniluracil; aldophosphamide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK7 polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); cyclophosphamide; thiotepa; taxoids and taxanes, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® docetaxel (Rhône-Poulenc Rorer, Antony, France); chloranbucil; gemcitabine (GEMZAR®); 6-thioguanine; mercaptopurine; platinum; platinum analogs or platinum-based analogs such as cisplatin, oxaliplatin and carboplatin; vinblastine (VELBAN®); etoposide (VP-16); ifosfamide; mitoxantrone; vincristine (ONCOVIN®); vinca alkaloid; vinorelbine (NAVELBINE®); novantrone; edatrexate; daunomycin; aminopterin; xeloda; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

An “antimetabolite chemotherapeutic agent” is an agent which is structurally similar to a metabolite, but can not be used by the body in a productive manner. Many antimetabolite chemotherapeutic agents interfere with the production of the nucleic acids, RNA and DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOME®), azocytosine, deoxycytosine, pyridmidene, fludarabine (FLUDARA®), cladrabine, 2-deoxy-D-glucose etc. The preferred antimetabolite chemotherapeutic agent is gemcitabine.

“Gemcitabine” or “2′-deoxy-2′,2′-difluorocytidine monohydrochloride (b-isomer)” is a nucleoside analogue that exhibits antitumor activity. The empirical formula for gemcitabine HCl is C9H11F2N3O4 A HCl. Gemcitabine HCl is sold by Eli Lilly under the trademark GEMZAR®.

A “platinum-based chemotherapeutic agent” comprises an organic compound which contains platinum as an integral part of the molecule. Examples of platinum-based chemotherapeutic agents include carboplatin, cisplatin, and oxaliplatinum.

By “platinum-based chemotherapy” is intended therapy with one or more platinum-based chemotherapeutic agents, optionally in combination with one or more other chemotherapeutic agents.

II. Modes of Carrying Out the Invention

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991).

1. Preparation of Agonist anti-CYTL1 Antibodies

The anti-CYTL1 antibodies can be produced by methods known in the art, including techniques of recombinant DNA technology.

i) Antigen Preparation

Soluble antigens or fragments thereof, optionally conjugated to other molecules, can be used as immunogens for generating antibodies. For transmembrane molecules, such as receptors, fragments of these (e.g., the extracellular domain of a receptor) can be used as the immunogen. Alternatively, cells expressing the transmembrane molecule can be used as the immunogen. Such cells can be derived from a natural source (e.g., cancer cell lines) or may be cells which have been transformed by recombinant techniques to express the transmembrane molecule. Other antigens and forms thereof useful for preparing antibodies will be apparent to those in the art.

(ii) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCI₂, or R₁N═C═NR, where R and R₁ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(iii) Monoclonal Antibodies

Monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subloned by limiting dilution procedures and grown by standard methods (Goding, MonoclonalAntibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

In a further embodiment, antibodies or antibody fragments can be isolated from antibody phage libraries generated using the techniques described in McCafferty et al., Nature, 348:552-554 (1990).

Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iv) Humanized and Human Antibodies

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad Sci. USA, 89:4285 (1992); Presta et al., J. Immnol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J.sub.H) gene in chimeric and germ-line mutant mice results in complete inhibifion of endogenous antibody production. Transfer of the human germ-line immunoglobulin gene array in such germ-line mutant mice will result in the production of human antibodies upon antigen challenge. See, e.g., Jakobovits et al, Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature, 355:258 (1992). Human antibodies can also be derived from phage-display libraries (Hoogenboom et al, J. Mol. Biol., 227:381 (1991); Marks et al, J. MoL Biol., 222:581-597 (1991); Vaughan et al., Nature Biotech., 14:309 (1996)). Generation of human antibodies from antibody phage display libraries is further described below.

(v) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods, 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology, 10:163-167 (1992)). In another embodiment as described in the example below, the F(ab′)₂ is formed using the leucine zipper GCN4 to promote assembly of the F(ab′)₂ molecule. According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

(vi) Multispecific Antibodies

Multispecific antibodies have binding specificities for at least two different epitopes, where the epitopes are usually from different antigens. While such molecules normally will only bind two different epitopes (i.e., bispecific antibodies, BsAbs), antibodies with additional specificities such as trispecific antibodies are encompassed by this expression when used herein.

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J, 10:3655-3659 (1991). According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in WO96/27011, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′-SH fragments can also be directly recovered from E. coli, and can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper pepbdes from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Nati. Acad. Sci. USA, 90:6444-6448 (1 993) has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al, J. Immunol., 152:5368 (1994).

Antibodies with more than two valencies are contemplated. For example, trispecific antibodies can be prepared. Tuft et al., J. Immunol., 147: 60 (1991).

(vii) Effector Function Engineering

It may be desirable to modify the antibody of the invention with respect to effector function, so as to enhance the effectiveness of the antibody. For example, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med., 176:1191-1195 (1992) and Shopes, B. J. Immunol., 148:2918-2922 (1992). Homodimeric antibodies with enhanced anti-tumor activity may also be prepared using heterobifunctonal cross-linkers as described in Wolff et al., Cancer Research, 53:2560-2565 (1993). Alternatively, an antibody can be engineered which has dual Fc regions and may thereby have enhanced complement lysis and ADCC capabilities. See Stevenson et al Anti-Cancer Drug Design 3:219-230 (1989).

(viii) Antibody-Salvage Receptor Binding Epitope Fusions.

In certain embodiments of the invention, it may be desirable to use an antibody fragment, rather than an intact antibody, to increase tumor penetration, for example. In this case, it may be desirable to modify the antibody fragment in order to increase its serum half life. This may be achieved, for example, by incorporation of a salvage receptor binding epitope into the antibody fragment (e.g., by mutation of the appropriate region in the antibody fragment or by incorporating the epitope into a peptide tag that is then fused to the antibody fragment at either end or in the middle, e.g., by DNA or peptide synthesis).

The salvage receptor binding epitope preferably constitutes a region wherein any one or more amino acid residues from one or two loops of a Fc domain are transferred to an analogous position of the antibody fragment. Even more preferably, three or more residues from one or two loops of the Fc domain are transferred. Still more preferred, the epitope is taken from the CH2 domain of the Fc region (e.g., of an IgG) and transferred to the CH1, CH3, or V.sub.H region, or more than one such region, of the antibody. Alternatively, the epitope is taken from the CH2 domain of the Fc region and transferred to the CL region or VL region, or both, of the antibody fragment.

(ix) Other Covalent Modifications of Antibodies

Covalent modifications of antibodies are included within the scope of this invention. They may be made by chemical synthesis or by enzymatic or chemical cleavage of the antibody, if applicable. Other types of covalent modifications of the antibody are introduced into the molecule by reacting targeted amino acid residues of the antibody with an organic derivatizing agent that is capable of reacting with selected side chains or the N- or C-terminal residues. Examples of covalent modifications are described in U.S. Pat. No. 5,534,615, specifically incorporated herein by reference. A preferred type of covalent modification of the antibody comprises linking the antibody to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

(x) Generation of Antibodies From Synthetic Antibody Phage Libraries

In a preferred embodiment, the invention provides a method for generating and selecting novel antibodies using a unique phage display approach. The approach involves generation of synthetic antibody phage libraries based on single framework template, design of sufficient diversities within variable domains, display of polypeptides having the diversified variable domains, selection of candidate antibodies with high affinity to target the antigen, and isolation of the selected antibodies.

Details of the phage display methods can be found, for example, WO03/102157 published Dec. 11, 2003, the entire disclosure of which is expressly incorporated herein by reference.

In one aspect, the antibody libraries used in the invention can be generated by mutating the solvent accessible and/or highly diverse positions in at least one CDR of an antibody variable domain. Some or all of the CDRs can be mutated using the methods provided herein. In some embodiments, it may be preferable to generate diverse antibody libraries by mutating positions in CDRH1, CDRH2 and CDRH3 to form a single library or by mutating positions in CDRL3 and CDRH3 to form a single library or by mutating positions in CDRL3 and CDRH1, CDRH2 and CDRH3 to form a single library.

A library of antibody variable domains can be generated, for example, having mutations in the solvent accessible and/or highly diverse positions of CDRH1, CDRH2 and CDRH3. Another library can be generated having mutations in CDRL1, CDRL2 and CDRL3. These libraries can also be used in conjunction with each other to generate binders of desired affinities. For example, after one or more rounds of selection of heavy chain libraries for binding to a target antigen, a light chain library can be replaced into the population of heavy chain binders for further rounds of selection to increase the affinity of the binders.

Preferably, a library is created by substitution of original amino acids with variant amino acids in the CDRH3 region of the variable region of the heavy chain sequence. The resulting library can contain a plurality of antibody sequences, wherein the sequence diversity is primarily in the CDRH3 region of the heavy chain sequence.

In one aspect, the library is created in the context of the humanized antibody 4D5 sequence, or the sequence of the framework amino acids of the humanized antibody 4D5 sequence. Preferably, the library is created by substitution of at least residues 95-100a of the heavy chain with amino acids encoded by the DVK codon set, wherein the DVK codon set is used to encode a set of variant amino acids for every one of these positions. An example of an oligonucleotide set that is useful for creating these substitutions comprises the sequence (DVK)₇. In some embodiments, a library is created by substitution of residues 95-100a with amino acids encoded by both DVK and NNK codon sets. An example of an oligonucleotide set that is useful for creating these substitutions comprises the sequence (DVK)₆ (NNK). In another embodiment, a library is created by substitution of at least residues 95-100a with amino acids encoded by both DVK and NNK codon sets. An example of an oligonucleotide set that is useful for creating these substitutions comprises the sequence (DVK)₅ (NNK). Another example of an oligonucleotide set that is useful for creating these substitutions comprises the sequence (NNK)₆. Other examples of suitable oligonucleotide sequences can be determined by one skilled in the art according to the criteria described herein.

In another embodiment, different CDRH3 designs are utilized to isolate high affinity binders and to isolate binders for a variety of epitopes. The range of lengths of CDRH3 generated in this library is 11 to 13 amino acids, although lengths different from this can also be generated. H3 diversity can be expanded by using NNK, DVK and NVK codon sets, as well as more limited diversity at N and/or C-terminal.

Diversity can also be generated in CDRH1 and CDRH2. The designs of CDR-H1 and H2 diversities follow the strategy of targeting to mimic natural antibodies repertoire as described with modification that focus the diversity more closely matched to the natural diversity than previous design.

For diversity in CDRH3, multiple libraries can be constructed separately with different lengths of H3 and then combined to select for binders to target antigens. The multiple libraries can be pooled and sorted using solid support selection and solution sorting methods as described previously and herein below. Multiple sorting satrategies may be employed. For example, one variation involves sorting on target bound to a solid, followed by sorting for a tag that may be present on the fusion polypeptide (e.g., anti-gD tag) and followed by another sort on target bound to solid. Alternatively, the libraries can be sorted first on target bound to a solid surface, the eluted binders are then sorted using solution phase binding with decreasing concentrations of target antigen. Utilizing combinations of different sorting methods provides for minimization of selection of only highly expressed sequences and provides for selection of a number of different high affinity clones.

High affinity binders for the target antigen can be isolated from the libraries. Limiting diversity in the H1/H2 region decreases degeneracy about 10⁴ to 10⁵ fold and allowing more H3 diversity provides for more high affinity binders. Utilizing libraries with different types of diversity in CDRH3 (e.g., utilizing DVK or NVT) provides for isolation of binders that may bind to different epitopes of a target antigen.

Of the binders isolated from the pooled libraries as described above, it has been discovered that affinity may be further improved by providing limited diversity in the light chain. Light chain diversity is generated in this embodiment as follows in CDRL1: amino acid position 28 is encoded by RDT; amino acid position 29 is encoded by RKT; amino acid position 30 is encoded by RVW; amino acid position 31 is encoded by ANW; amino acid position 32 is encoded by THT; optionally, amino acid position 33 is encoded by CTG; in CDRL2: amino acid position 50 is encoded by KBG; amino acid position 53 is encoded by AVC; and optionally, amino acid position 55 is encoded by GMA; in CDRL3: amino acid position 91 is encoded by TMT or SRT or both; amino acid position 92 is encoded by DMC; amino acid position 93 is encoded by RVT; amino acid position 94 is encoded by NHT; and amino acid position 96 is encoded by TWT or YKG or both.

In another embodiment, a library or libraries with diversity in CDRH1, CDRH2 and CDRH3 regions is generated. In this embodiment, diversity in CDRH3 is generated using a variety of lengths of H3 regions and using primarily codon sets XYZ and NNK or NNS. Libraries can be formed using individual oligonucleotides and pooled or oligonucleotides can be pooled to form a subset of libraries. The libraries of this embodiment can be sorted against target bound to solid. Clones isolated from multiple sorts can be screened for specificity and affinity using ELISA assays. For specificity, the clones can be screened against the desired target antigens as well as other nontarget antigens. Those binders to the target antigen can then be screened for affinity in solution binding competition ELISA assay or spot competition assay. High affinity binders can be isolated from the library utilizing XYZ codon sets prepared as described above. These binders can be readily produced as antibodies or antigen binding fragments in high yield in cell culture.

In some embodiments, it may be desirable to generate libraries with a greater diversity in lengths of CDRH3 region. For example, it may be desirable to generate libraries with CDRH3 regions ranging from about 7 to 19 amino acids.

High affinity binders isolated from the libraries of these embodiments are readily produced in bacterial and eukaryotic cell culture in high yield. The vectors can be designed to readily remove sequences such as gD tags, viral coat protein component sequence, and/or to add in constant region sequences to provide for production of full length antibodies or antigen binding fragments in high yield.

A library with mutations in CDRH3 can be combined with a library containing variant versions of other CDRs, for example CDRL1, CDRL2, CDRL3, CDRH1 and/or CDRH2. Thus, for example, in one embodiment, a CDRH3 library is combined with a CDRL3 library created in the context of the humanized 4D5 antibody sequence with variant amino acids at positions 28, 29, 30, 31, and/or 32 using predetermined codon sets. In another embodiment, a library with mutations to the CDRH3 can be combined with a library comprising variant CDRH1 and/or CDRH2 heavy chain variable domains. In one embodiment, the CDRH1 library is created with the humanized antibody 4D5 sequence with variant amino acids at positions 28, 30, 31, 32 and 33. A CDRH2 library may be created with the sequence of humanized antibody 4D5 with variant amino acids at positions 50, 52, 53, 54, 56 and 58 using the predetermined codon sets.

(xi) Antibody Mutants

To generate an antibody mutant, one or more amino acid alterations (e.g., substitutions) are introduced in one or more of the hypervariable regions of the parent antibody. Alternatively, or in addition, one or more alterations (e.g., substitutions) of framework region residues may be introduced in the parent antibody where these result in an improvement in the binding affinity of the antibody mutant for the antigen from the second mammalian species. Examples of framework region residues to modify include those which non-covalently bind antigen directly (Amit et al. (1986) Science 233:747-753); interact with/effect the conformation of a CDR (Chothia et al. (1987) J. Mol. Biol. 196:901-917); and/or participate in the V_{L}-V_{H} interface (EP 239 400B1). In certain embodiments, modification of one or more of such framework region residues results in an enhancement of the binding affinity of the antibody for the antigen from the second mammalian species. For example, from about one to about five framework residues may be altered in this embodiment of the invention. Sometimes, this may be sufficient to yield an antibody mutant suitable for use in preclinical trials, even where none of the hypervariable region residues have been altered. Normally, however, the antibody mutant will comprise additional hypervariable region alteration(s).

The hypervariable region residues which are altered may be changed randomly, especially where the starting binding affinity of the parent antibody is such that such randomly produced antibody mutants can be readily screened.

One useful procedure for generating such antibody mutants is called “alanine scanning mutagenesis” (Cunningham and Wells (1989) Science 244:1081-1085). Here, one or more of the hypervariable region residue(s) are replaced by alanine or polyalanine residue(s) to affect the interaction of the amino acids with the antigen from the second mammalian species. Those hypervariable region residue(s) demonstrating functional sensitivity to the substitutions then are refined by introducing further or other mutations at or for the sites of substitution. Thus, while the site for introducing an amino acid sequence variation is predetermined, the nature of the mutation per se need not be predetermined. The ala-mutants produced this way are screened for their biological activity as described herein.

Normally one would start with a conservative substitution such as those shown below under the heading of “preferred substitutions.” If such substitutions result in a change in biological activity (e.g., binding affinity), then more substantial changes, denominated “exemplary substitutions” in the following table, or as further described below in reference to amino acid classes, are introduced and the products screened.

Preferred Substitutions:

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

Even more substantial modifications in the antibodies biological properties are accomplished by selecting substitutions that differ significantly in their effect on maintaining: (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr, asn, gln;

(3) acidic: asp, glu;

(4) basic: his, lys, arg;

(5) residues that influence chain orientation: gly, pro; and

(6) aromatic: trp, tyr, phe.

Non-conservative substitutions will entail exchanging a member of one of these classes for another class.

In another embodiment, the sites selected for modification are affinity matured using phage display (see above).

Nucleic acid molecules encoding amino acid sequence mutants are prepared by a variety of methods known in the art. These methods include, but are not limited to, oligonucleotide-mediated (or site-directed) mutagenesis, PCR mutagenesis, and cassette mutagenesis of an earlier prepared mutant or a non-mutant version of the parent antibody. The preferred method for making mutants is site directed mutagenesis (see, e.g., Kunkel (1985) Proc. Natl. Acad. Sci. USA 82:488).

In certain embodiments, the antibody mutant will only have a single hypervariable region residue substituted. In other embodiments, two or more of the hypervariable region residues of the parent antibody will have been substituted, e.g. from about two to about ten hypervariable region substitutions.

Ordinarily, the antibody mutant with improved biological properties will have an amino acid sequence having at least 75% amino acid sequence identity or similarity with the amino acid sequence of either the heavy or light chain variable domain of the parent antibody, more preferably at least 80%, more preferably at least 85%, more preferably at least 90%, and most preferably at least 95%. Identity or similarity with respect to this sequence is defined herein as the percentage of amino acid residues in the candidate sequence that are identical (i.e., same residue) or similar (i.e., amino acid residue from the same group based on common side-chain properties, see above) with the parent antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity.

Following production of the antibody mutant, the biological activity of that molecule relative to the parent antibody is determined. As noted above, this may involve determining the binding affinity and/or other biological activities of the antibody. In a preferred embodiment of the invention, a panel of antibody mutants is prepared and screened for binding affinity for the antigen or a fragment thereof. One or more of the antibody mutants selected from this initial screen are optionally subjected to one or more further biological activity assays to confirm that the antibody mutant(s) with enhanced binding affinity are indeed useful, e.g. for preclinical studies.

The antibody mutant(s) so selected may be subjected to further modifications, oftentimes depending on the intended use of the antibody. Such modifications may involve further alteration of the amino acid sequence, fusion to heterologous polypeptide(s) and/or covalent modifications such as those elaborated below. With respect to amino acid sequence alterations, exemplary modifications are elaborated above. For example, any cysteine residue not involved in maintaining the proper conformation of the antibody mutant also may be substituted, generally with serine, to improve the oxidative stability of the molecule and prevent aberrant cross linking. Conversely, cysteine bond(s) may be added to the antibody to improve its stability (particularly where the antibody is an antibody fragment such as an Fv fragment). Another type of amino acid mutant has an altered glycosylation pattern. This may be achieved by deleting one or more carbohydrate moieties found in the antibody, and/or adding one or more glycosylation sites that are not present in the antibody. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The same type of procedures and alterations can be used to create CYTL1 variants that act as agonists of a native-sequence CYTL1 polypeptide.

(xii) Recombinant Production of Antibodies and Other Polypeptides

For recombinant production of an antibody, the nucleic acid encoding it is isolated and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence (e.g., as described in U.S. Pat. No. 5,534,615, specifically incorporated herein by reference).

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serrafia, e.g., Serratia marcescans, and Shigeila, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One preferred E. coli cloning host is E. Coli 294 (ATCC 31,446), although other strains such as E. coli B, E. Coli X 1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subloned for growth in suspension culture, Graham et al, J. Gen Virol., 36:59 (1977)); baby hamster kidney cells (BHK, ATCC CCL 10); Chinese hamster ovary cells/-DHFR (CHO, Urlaub et al., Proc. Natl. Acad. Sci. USA, 77:4216 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce the antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz., 58:44 (1979), Barnes et al., Anal. Biochem., 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed cells, is removed, for example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human .gamma.1, .gamma.2, or .gamma.4 heavy chains (Lindmark et al., J. Immunol. Meth., 62:1-13 (1983)). Protein G is recommended for all mouse isotypes and for human .gamma.3 (Guss et al., EMBO J., 5:1567-1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH 3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipiation are also available depending on the antibody to be recovered.

Other recombinant polypeptides, such as CYTL1 and CYTL1 variants, can be prepared by similar procedures.

2. Screening for Agonist Anti-CYTL1 Antibodies or Other Agonists of CYTL1

Agonist antibodies and other agonists of CYTL1 can be identified in traditional (competitive) binding assays or activity assays.

Screening assays for CYTL1 agonists may be designed to identify compounds that bind or complex with a uPAR, or otherwise interfere with the interaction of CYTL1 and uPAR, and are capable of competitively inhibit the binding of uPA to uPAR. The screening assays provided herein include assays amenable to high-throughput screening of chemical libraries, making them particularly suitable for identifying small molecule drug candidates. Generally, binding assays and activity assays are provided.

The assays can be performed in a variety of formats, including, without limitation, protein-protein binding assays, biochemical screening assays, immunoassays, and cell-based assays, which are well characterized in the art.

All assays are common in that they call for contacting a candidate CYTL1 agonist with uPA, uPAR and CYTL1 under conditions and for a time sufficient to allow these two components to interact. In binding assays, the interaction is binding, and the complex formed can be isolated or detected in the reaction mixture. In a particular embodiment, either the uPA or uPAR polypeptide or the candidate agonist is immobilized on a solid phase, e.g., on a microtiter plate, by covalent or non-covalent attachments. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

Preferably, the CYTL1 antagonists are identified or further tested based on their ability to inhibit a uPA biological activity, such as, for example, the ability of uPA to mediate tumor formation or metastasis or to induce or support angiogenesis, including, but not limited to, tumor angiogenesis.

It is emphasized that the screening assays specifically discussed herein are for illustration only. A variety of other assays, which can be selected depending on the type of the antagonist candidates screened (e.g. polypeptides, peptides, non-peptide small organic molecules, nucleic acid, etc.) are well know to those skilled in the art and are equally suitable for the purposes of the present invention.

3. Pharmaceutical Compositions

CYTL1 and CYTL1 agonists, including agonist CYTL1 antibodies, can be administered for the treatment of various disorders associated with the production or function of uPA, including various types or tumor or cancer, including metastasis or tumor or cancer and diseases and disorders characterized by unwanted angiogenesis, including, without limitation, angiogenesis of tumor and cancer, in the form of pharmaceutical compositions.

Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993).

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The formulation herein may also contain more than one active compound as necessary for the particular indication being treated, preferably those with complementary activities that do not adversely affect each other. Such molecules are suitably present in combination in amounts that are effective for the purpose intended, or may be formulated separately, and administered concurrently or consecutively, in any order.

For example, the CYTL1 and the CYTL1 agonists of the present invention may be administered in combination with one or more additional therapeutic agents, such as anti-angiogenic agents, an anti-neoplastic compositions, chemotherapeutic agents and/or cytotoxic agents.

The following examples are offered for illustrative purposes only, and are not intended to limit the scope of the present invention in any way.

All patent and literature references cited in the present specification are hereby expressly incorporated by reference in their entirety.

### EXAMPLE

### Materials and Methods

Materials—Recombinant human uPAR, uPA and uPA-ATF were obtained from R&D Systems (Minneapolis, Minn.). Pro-uPA was obtained from Cortex Biochem (San Leandro, Calif.). Low molecular weight heparin from porcine mucosa was purchased from Sigma Chemical Company (St. Louis, Mo.).

In-situ hybridization—For thin section in-situ hybridization, PCR primers were designed to amplify a 449 bp fragment of mouse CYTL1 spanning from nt. 111-560 of NM_{—}001081106 [5′-CCCACCTGCTACTCTCGGATG-3′ (SEQ ID NO: 3) and 5′-GGCAGGTCTAACAGTGGCACTAA-3′ (SEQ ID NO: 4)] or a 431 bp fragment of human CYTL1 spanning from nt. 94-525 of NM_018659 [5′-TCCCCCGACCTGCTACTC-3′ (SEQ ID NO: 5) and 5′-CCTGTGAGGGTCATGGCTCTGGC-3′ (SEQ ID NO: 6)]. Primers included extensions encoding 27-nucleotide T7 or T3 RNA polymerase initiation sites to allow in vitro transcription of sense or antisense probes, respectively, from the amplified products.

Formalin-fixed, paraffin-embedded 5 μm sections were deparaffinized, deproteinated in 4 μg/ml Proteinase K for 30 min at 37° C., and further processed for in situ hybridization as previously described (Jubb et al., (2006)Methods in Molecular Biology 326, 255-264). ³³P-UTP labeled sense and antisense probes were hybridized to the sections at 55° C. overnight. Unhybridized probe was removed by RNase treatment and stringent washing. The slides were dehydrated through graded ethanols, dipped in NTB nuclear track emulsion (Eastman Kodak), exposed for 4 weeks at 4° C. then developed and counterstained with hematoxylin and eosin.

ATDC5 cell maturation assay—ATDC5 cells were obtained from RIKEN Cellbank, Japan, and maintained in a 50:50 mix of Dulbecco's modified Eagle's medium (DMEM) and Hams F12 medium, supplemented with 5% fetal bovine serum and 2 mM L-glutamine. Cells were seeded at 10⁵ cells per well in 12-well plates and grown to confluence, then treated with 10 μg/ml recombinant bovine insulin and 50 μg/ml ascorbic acid to induce chondrocyte differentiation. RNA was harvested from triplicate wells at various timepoints during maturation. At day 11, some cells were treated with 0.1 ng/ml IL-1β, and RNA harvested after a further 24 hours.

Collagen-induced athritis—Male DBA/1J mice, 7-8 weeks old, were obtained from The Jackson Laboratory (Bar Harbor, Me.) and maintained in accordance with American Association of Laboratory Animal Care guidelines. All experimental procedures were approved by the Institutional Animal Care and Use Committee at Genentech. Collagen-induced arthritis was induced following a standard protocol (Barck et al. (2004) Arthritis and rheumatism 50(10), 3377-3386) with injections of bovine collagen type II in complete Freund's adjuvant at day 0 and day 21. Total RNA was extracted from footpads with clinical disease, and from footpads of healthy DBA/1J control mice.

GeneLogic BioExpress Data—A commercially available database, BioExpress (GeneLogic, Gaithersburg, Md.), was used to query gene expression during collagen-induced arthritis. Samples had been analyzed on Affymetrix U133A GeneChip following the manufacturer's protocols (Affymetrix, Santa Clara, Calif., USA).

Expression and purification of CYTL1—-Human CYTL1 with an N-terminal poly-histidine-tag was expressed in insect cells using the FastBac baculoviral system (Invitrogen, Carlsbad, Calif.), with a honey-bee melittin signal peptide. The protein was purified over a Ni⁺⁺-Sepharose 6 FF column (Amersham Biosciences, Piscataway, N.J.). The tag was cleaved with recombinant enterokinase (Novagen, Madison, Wis.), and tag and enzyme were depleted using Ni⁺⁺-Sepharose and EKapture agarose (Novagen). CYTL1 was further purified by salt-gradient-elution from a heparin column (HiTrap His, Amersham), then concentrated using Centricon centrifugal concentrators (Millipore, Billerica, Mass.) with a 3 kDa MW cut-off. The filtration flow-through was reserved for use as buffer control in surface plasmon resonance and cell-based assays.

Expression Cloning—An expression library containing approximately 14,000 full-length human cDNAs in pCMV-SPORT-based vectors was obtained from Origene (Rockville, Md.). Human placental alkaline phosphatase (AP) fusion proteinsl; TACI-AP and human and murine CYTL1-AP, were expressed in 293 cells by transient transfection using pRK5-based vectors. Cell-conditioned media were assayed for enzymatic activity and diluted as necessary to equalize activity. COS cells in 24-well plates were transfected with pools of expression constructs using FuGene6 (Roche). After 48 hours, the adherent cells were washed then incubated for 30 min at room temperature with cell-conditioned medium containing CYTL1-AP or TACI-AP, supplemented with 0.1% BSA. After washing, bound fusion protein was cross-linked to the surface by incubation with 10% neutral-buffered formalin. Endogenous alkaline phosphatase was inactivated by incubating plates at 65° C. for 90 min, then alkaline-phosphatase activity was detected with Western Blue Stabilized Substrate (Promega, Madison, Wis.).

Inactivation of uPA—Protease activity of uPA was irreversibly inhibited by treatment with 10 mM di-isopropyl fluorophosphate (DFP) for 2 hours at room temperature. Excess DFP was removed by extensive dialysis. Complete inactivation was confirmed using the uPA substrate S-2444 (diaPharma, West Chester, Ohio).

Cell-surface binding assays—COS or CHO cells, as indicated in figure legend, were transfected with expression vector encoding full-length human or mouse uPAR. After 24 hours, transfectants were transferred to 24-well plates. After a further 24 hours, cells were washed, then probed with cell-condititioned media containing CYTL1-AP as detailed under ‘Expression cloning.’ For quantitation of bound alkaline-phosphatase activity, BluePhos (KPL, Gaithersburg, Md.) was used as the detection reagent. For competition assays, the indicated concentrations of pro-uPA, uPA-ATF, DFP-uPA or purified CYTL1 were added to the CYTL1-AP cell-conditioned media immediately prior to incubation with uPAR-transfected cells.

RNA extraction and quantitative Real-Time PCR (qRT-PCR)—Total RNA was extracted using RNeasy miniprep kits (Qiagen, Germantown, Md.), quantified by optical density, and reverse-transcribed with Omniscript RT (Qiagen). The cDNA generated was assayed in duplicate using QuantiTect probe in a 7500 Real Time PCR System (Applied Biosystems, Foster City, Calif.). The amplified signal was normalized against that of β-actin or GAPDH as indicated. Primers and probes are listed in Table 1.

Surface Plasmon Resonance—Surface Plasmon Resonance experiments were performed with a BIAcore 3000 (BIAcore Inc., Uppsala, Sweden), using HBS-P buffer (50 mM HEPES pH7.4, 150 mM NaCl, 0.005% surfactant) at a flow-rate of 10 μl/min. Recombinant human uPAR was immobilized on a CM5 sensor chip using standard amine-coupling chemistry. Briefly, the surface was activated with an 8-minute injection of freshly mixed N-hydroxysuccinimide and 1-ethyl-3-(3-dim-ethylaminopropyl)-carbodiimide hydrochloride, uPAR was injected at 20 μg/ml in 10 mM sodium acetate pH4.5, and unreacted esters were quenched with 1M ethanolamine. Empty flow-cells and flow-cells with BSA coupled were used as controls. For equilibrium binding analysis, various concentrations of purified CYTL1 and equivalent dilutions of buffer were injected over all flow-cells. The response to buffer alone was subtracted from the CYTL1 response, and peak response at equilibrium was recorded. Dilutions and measurements were performed three times. BIAEval software (BIAcore) was used to fit the equilibrium binding data using the Langmuir equation. To demonstrate competition between CYTL1 and pro-uPA, CYTL1 was injected into a uPAR-coupled flowcell before and immediately after injection of a saturating concentration of pro-uPA.

Secondary structure prediction—CYTL1 species homologues were identified from ESTs and genomic sequences by BLAST and BLAT searches, and aligned using ClustalX. The multiple-species alignment was used as input to the Jnet neural-network-based algorithm (33) via the JPred website, to generate the secondary structure prediction. Source sequences: HumanNM 018659,AY359101; Chimp (Pan Troglodytes) Genome prediction: GenScan chr4_{—}2.3; Macaque (Rhesus macaque, Macaca mulatta) Genome prediction: Genscan chr5.4.000.a; Bovine (Bos taurus) BE683329 BE683328 BE683327; Mouse NM_{—}001081106; Rat CK476529,CK363591,CK359536; Possum (Monodelphis domestica) Genome prediction: GenScan c_{—}619.34; Chicken (Gallus gallus) BX931297, Xenopus (laevis) EB474543,EB474068,EC277031; P oliv (Bastard Halibut, Paralichthys olivaceus) CX285777,CX285687; Halibut (Hippoglossus hippoglossus) EB036635; Fugu (rubripes) Genomic sequence identified by BLAT—chrUn:189,159,975-189,160,957; Medaka (Oryzias latipes) AU241696,AU241459,AU177768; Salmon (Salmo salar) EG847194,EG833037,EG802555; Trout (Oncorhynchus mykiss) BX889777,BX866010; Smelt-Osmerus mordax EL55175; Minnow (Pimephales promelas) DT309382,DT347709,DT106962; Z-fish (Zebrafish, Danio rerio) EE214359,EE213198,EB957495; Z-fish2 (Danio rerio—second CYTL1 homologue) AL913033,EH466946,EH441042,AL913034.

### RESULTS

In vivo expression of CYTL1—-Whole-mount in situ hybridization reveals CYTL1 expression in a wide variety of developing skeletal elements in the mouse embryo (FIG. 1), including ribs, vertebrae and long-bones, at stages corresponding to the cartilage anlagen. Thin section in situ hybridization reveals expression of CYTL1 predominantly in cartilage, with particularly high expression in the superficial layer of articular cartilage. CYTL1 expression is also seen in the cartilage rings of the trachea. High levels of expression are seen in the tendon sheath, around the site of contact between tendon and bone. CYTL1 expression is observed in hypertrophic chondrocytes of the developing long-bones, and also in arterial (but not venous) endothelial cells, detected first in the aorta at embryonic d17, and spreading throughout the muscular arteries in adult mice.

The timecourse of expression of CYTL1 during chondrocyte maturation was studied using the clonal mouse pre-chondrocyte cell-line ATDC5 (FIG. 2). ATDC5 cells differentiate in response to insulin, with well-characterized changes in gene-expression. CYTL1 was not detectable in undifferentiated cells, but following initiation of differentiation, CYTL1 expression was detected by day 4, along with expression of the early chondrocyte marker collagen II. CYTL1 expression continued to increase to day 12, along with increased expression of aggrecan, a marker for mature chondrocytes.

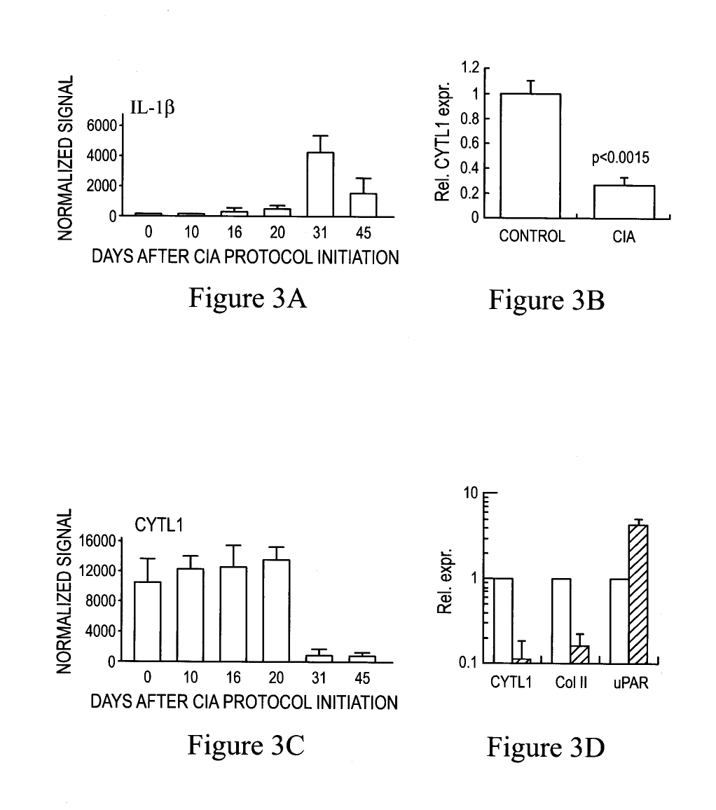

Expression Modulation—Because many cytokines play critical regulatory roles in inflammation (Heinrich et al. (2003) Biochem. J. 374(Pt 1), 1-20), we investigated CYTL1 expression during the course of a mouse model of experimental arthritis disease. Microarray data from a collagen-induced arthritis (CIA) model indicate that CYTL1 expression in mouse joints is downregulated in disease, coincident with the peak of clinical score and production of inflammatory cytokines, particularly IL-1β (FIG. 3a). Quantitative RT-PCR analysis of RNA extracted from mouse footpads in a separate experiment confirmed the downregulation of CYTL1 expression in CIA (FIG. 3b). IL-1β plays a key role in the progression of CIA (Williams, R. O. (2004) Methods in Molecular Medicine 98, 207-216), is a critical mediator of cartilage destruction (Zwerina et al. (2007) Proc. Natl. Acad. Sci. USA, 104(28), 11742-11747), and acts directly on chondrocytes to induce downregulation of several chondrocyte maturation-related genes (LeFebvre et al. (1990) Biochimica et biophysica acta 1052(3), 366-378). To test whether CYTL1 downregulation in CIA could be explained by direct action of IL-1β, we exposed differentiated ATDC5 chondrocytes to IL-1β and assayed gene expression by qRT-PCR. We observed a 10-fold reduction in CYTL1 expression in response to 0.1 ng/ml IL-1β (FIG. 3c). Conversely, uPAR expression was upregulated by IL-1β treatment.

Identification of a Receptor—CYTL1 has been earlier described as a four-helical cytokine based on the secondary structure prediction of several amphipathic α-helices (Liu et al. (2000) Genomics 65(3), 283-292). For a more thorough fold analysis, we first generated an evolutionarily diverse collection of all known CYTL1 orthologs by exhaustive PsiBLAST queries of GenBANK (Altschul et al. (1997) Nucl. Acids Res. 25(17), 3389-3402), aligned the sequences with ClustalX (Thompson et al. (1997) Nucl. Acids Res. 25(24), 4876-4882) and then used both PsiPRED and JNet algorithms (Jones, D. T. (1999) J Mol Biol 292(2), 195-202; Cuff, J. A., and Barton, G. J. (2000) Proteins 40(3), 502-511) to generate an accurate secondary structure prediction (Supplemental FIG. 1). Three alpha-helices with lengths and spacings consistent with helices A through C of a short-chain cytokine (Rozwarski et al. (1994) Structure 2(3), 159-173) are reliably predicted, along with an additional single-turn of α-helix and two short β-strands as seen in the short chain cytokines like GM-CSF, IL-3 and IL-13. The relationship between CYTL1 and the four-helical cytokine family is strengthened by analysis of its genomic organization. Four-helical cytokines show a stereotypical positioning of exon boundaries relative to structural features, with the junctions all in phase 0, characteristics shared by CYTL1. However, in its predicted secondary structure, CYTL1 departs dramatically from the helical cytokine pattern in its C-terminal region where the fourth or D-helix appears to be absent or severely truncated. As this helix is critical for binding of helical cytokines to their high-affinity receptors (Clackson, T., and Wells, J. A. (1995) Science (New York, N.Y. 267(5196), 383-386; Boulanger, M. J., and Garcia, K. C. (2004) Advances in Protein Chemistry 68, 107-146), this suggested that CYTL1 may have adopted a divergent mode of receptor binding, perhaps defecting to an unrelated receptor family.

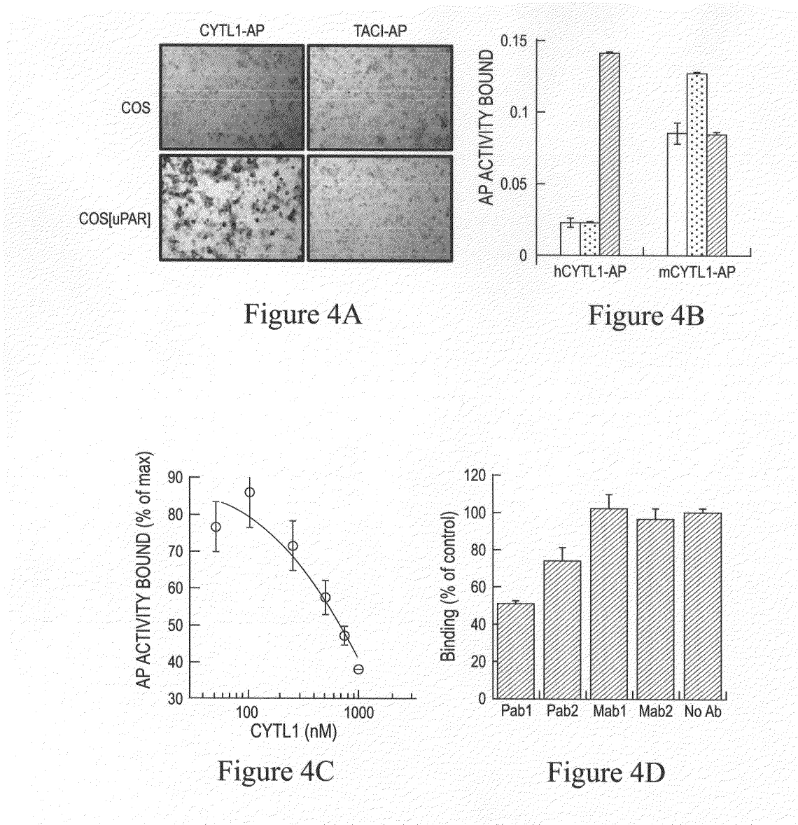

We undertook an unbiased search for a receptor for CYTL1 using an expression-cloning approach. A CYTL1-alkaline phosphatase fusion protein (CYTL1-AP) was used to probe COS cells transfected with pooled expression constructs from a library of full-length human clones. We identified a single pool which conferred upon cells the ability to bind CYTL1-AP, then screened the individual clones constituting this pool to identify a single clone responsible (FIG. 4a). Sequencing of this clone revealed that it encoded uPAR. Expression of uPAR conferred no binding for a control alkaline-phosphatase fusion protein, TACI-AP (Transmembrane Activator Calcium modulator and cyclophilin ligand Interactor), used at an equivalent concentration to CYTL1-AP, as measured by enzymatic activity. Similar results were observed on CHO cells. Expression of C4.4A, structurally related to uPAR, did not confer the ability to bind CYTL1-AP (data not shown). COS cells transfected with murine uPAR were capable of binding mouse CYTL1-AP fusion protein (FIG. 4b), indicating evolutionary conservation of the interaction between CYTL1 and uPAR. Little or no cross-species binding was observed.

An excess of purified CYTL1 was capable of inhibiting the binding of CYTL1-AP to uPAR-transfected cells in a concentration-dependent manner (FIG. 4c). Assuming homologous competition, the affinity of CYTL1 for uPAR at the cell-surface from three independent experiments is approximately 1.5 μM±0.5 μM (SEM).

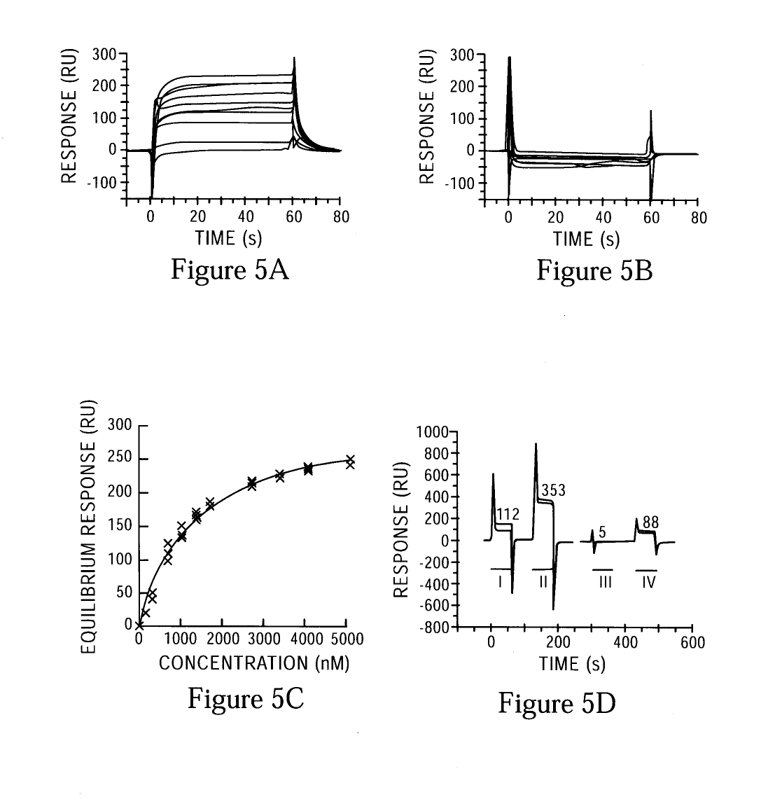

uPAR has been shown to interact with a number of cell-surface and extracellular proteins, including uPA, αᵥ and β₁ integrins, vitronectin, uPAR-Associated Protein (uPARAP/Endo180) and IGF-IIR (Ploug, M. (2003) Current Pharmaceutical Design 9(19), 1499-1528). To assess whether CYTL1 interacts directly with uPAR, and without a requirement for accessory proteins, we carried out surface plasmon resonance (SPR) analysis using the BIAcore 3000. Purified CYTL1 bound specifically to immobilized uPAR (FIG. 5). Similar binding was also observed with CYTL1 expressed in mammalian cells and bacteria (data not shown). The kinetics of the interaction are extremely rapid, and could not be accurately quantified. Equilibrium-binding measurements were obtained by injecting varying concentrations of CYTL1 over immobilized uPAR and over control flow-cells (FIG. 5c). The Langmuir equation for single-site binding fits the data well, providing a measure of the affinity constant for the interaction of CYTL1 and uPAR of 1.1 μM±0.06 μM. No difference in affinity was observed when the density of uPAR immobilized was varied over a five-fold range.

Glycosaminoglycans are known to significantly affect the binding of several cytokines to their receptors. Our purification of CYTL1 protein made use of an interaction between CYTL1 and heparin, so we sought to further characterize this interaction. CYTL1 and heparin, alone and in combination, were injected over immobilized uPAR (FIG. 5d). Heparin and CYTL1 alone each gave transient binding (88RU and 112RU); when CYTL1 and heparin were injected together, a greater-than-additive effect (353RU) was observed, indicating that heparin enhances the binding of CYTL1 to uPAR.

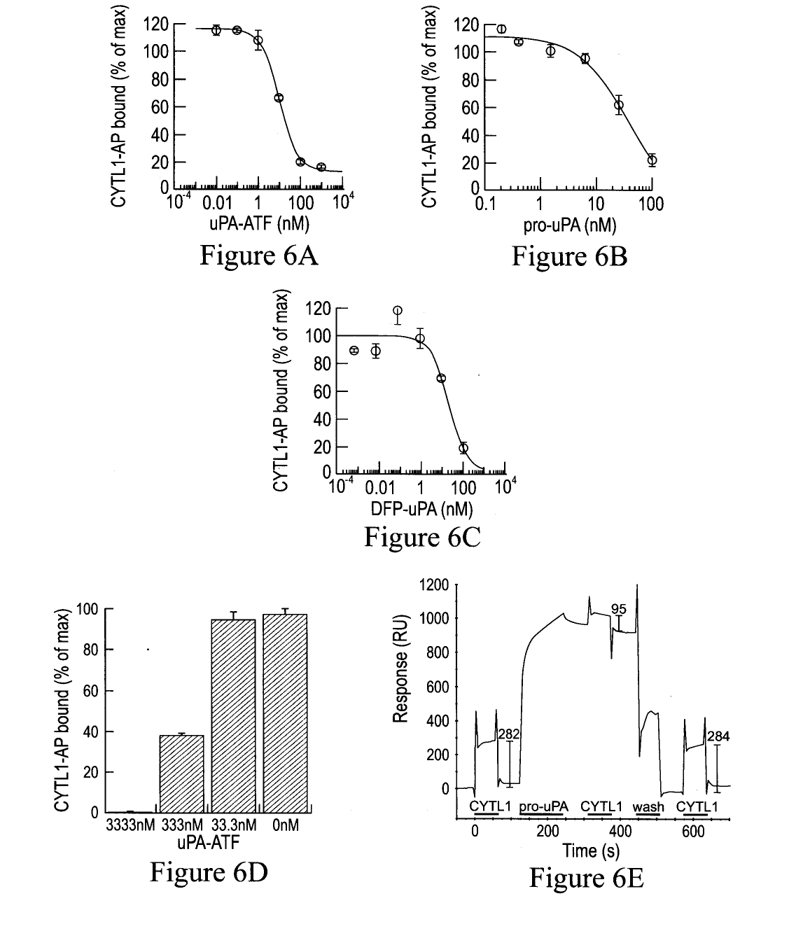

Competitive binding—uPAR is a cell-surface receptor for uPA. To establish whether CYTL1 competes with uPA for binding to uPAR, cells transfected with uPAR were incubated with CYTL1AP in the presence of varying concentrations of the proteolytically inactive pro-enzyme (pro-uPA), the receptor-binding amino terminal fragment (uPA-ATF) or full-length uPA inactivated by treatment with diisopropyl fluorophosphate (DFP-uPA). All three forms of uPA competed with CYTL1-AP for binding to the surface of uPAR-expression cells (FIG. 6a-c), with 50% inhibition of binding achieved with between 10 nM and 40 nM of competitor. To test whether this competition occurs in the absence of additional proteins, and in the absence of the bulky alkaline-phosphatase tag, SPR analysis was again used (FIG. 6d). CYTL1 was injected over immobilized uPAR, giving 282 response units (RU) of binding. pro-uPA was then injected at a high concentration to achieve over 50% receptor occupancy. A second injection of CYTL1 gave greatly diminished binding (95R), indicating that receptor occupancy by pro-uPA reduces the amount of receptor available for binding to CYTL1. Following dissociation of pro-uPA from the uPAR by low pH, the ability to bind CYTL1 was restored.

### DISCUSSION

CYTL1 is a secreted protein produced in a tissue-restricted manner, primarily by chondrocytes and arterial endothelial cells. In its predicted secondary structure and genomic organization, CYTL1 retains a partial resemblance to a four-α-helix hemopoietic cytokine. With few exceptions, all members of this protein family bind to an evolutionarily conserved family of cell-surface receptors distinguished by a conserved pair of fibronectin type-3 (Fn3)-like cytokine-binding modules (Bazan, J. F. (1990) Proc. Natl. Acad. Sci. USA, 87(18), 6934-6938). These receptors associate through their intracellular extensions with protein tyrosine kinases of the JAK family and STAT transcription factors, through which a signal is triggered following receptor oligomerization driven by extracellular ligand binding. The three exceptions to this paradigm, M-CSF, SCF and Flt3L, have ‘defected’ to a second class of PDGFR-like receptors which bind their ligands with immunoglobulin (Ig)-like domains, and signal through cytoplasmic tyrosine kinase domains. No conventional cytokine receptors were identified in the screen for a receptor for CYTL1. The receptor identified, uPAR, is structurally unrelated to either of the aforementioned families, and instead is largely comprised of three Ly6-uPAR (LU)-like modules (Ploug, M., and Ellis, V. (1994) FEBS Lett 349(2), 163-168; Barinka et al. (2006) J Mol Biol 363(2), 482-495; Llinas et al. (2005) Embo J 24(9), 1655-1663)—a domain first identified structurally in snake neurotoxins—that is attached to the plasma membrane by a GPI anchor. No interactions between four-helical cytokines and LU-modular proteins have been documented, though fold-relatives of these domains comprise the cytokine-binding segments of the signaling receptors for TGF-β, activins and Bone Morphogenetic Proteins (BMPs) (Gyetko et al. (1994) J. Clin. Invest. 93(4), 1380-1387). If CYTL1 is indeed a four-helical cytokine then this represents an entirely novel class of interaction for the LU fold.

Cytokines typically exert their function by sequentially binding to a series of receptors to form a high-affinity signaling complex that triggers an intracellular phosphorylation cascade. It is not yet clear whether uPAR is a true signaling receptor for CYTL1 or whether it is more accurately described as a CYTL1-binding protein. Lacking intracellular and transmembrane domains, uPAR acting alone cannot function as a signaling receptor. However, a number of signaling pathways have been shown to be activated following ligation of uPAR, with activation of the protein kinases Src, JAK-1, Hck, FAK and ERK1/2 reported (Webb et al., J. Cell Biol., 152(4), 741-752; Tang et al. (1998) J Biol Chem 273(29), 18268-18272; Resnati et al., Embo J, 15(7), 1572-1582; Dumler et al. (1998) J. Biol. Chem., 273(1), 315-321). The mechanisms of signal transduction are still unclear, though a role for integrins has been strongly implicated. A direct and uPA-dependent interaction between uPAR and integrin α3β1 (Wei et al. (2001) Mol. Biol. Cell 12(10), 2975-2986), has been demonstrated, as has an indirect interaction, with vitronectin binding to uPAR in a uPA-dependent manner, and then ligating integrins (Madsen et al. (2007) J. Cell Biol. 177(5), 927-939). Binding of CYTL1 to uPAR could potentially result in intracellular signal transduction in a similar manner if CYTL1 can bridge uPAR with another transmembrane chain, or if it locally regulates the interaction of uPAR with integrins or other signaling molecules.

We have shown that CYTL1 binds to uPAR in competition with pro-uPA, uPA-ATF and DFP-inactivated uPA. CYTL1 can therefore function as an antagonist of the interaction of uPA with uPAR. The uPA-uPAR interaction serves to bring pro-uPA into contact with cell-surface activators such as hepsin and matriptase, which cleave pro-uPA to generate mature uPA. Binding to uPAR also enhances the activity of mature uPA. By competing for binding of uPAR, CYTL1 would reduce cell-associated uPA activity. The biological effects of competitive inhibitors of the uPA-uPAR interaction have been investigated in a number of systems. Tumor cells transfected with a proteolytically inactive mutant form of uPA show reduced capacity for metastasis (Crowly et al. (1993) Proc. Natl. Acad. Sci. USA, 90(11), 5021-5025), uPA-ATF inhibits tube-formation by microvascular endothelial cells (Croon et al. (1999) Am J Pathol 154(6), 1731-1742) and angiogenesis following retinal injury (Le Gat et al. (2003) Gene Ther 10(25), 2098-2103). By binding to uPAR in competition with uPA, cartilage-derived CYTL1 may mediate similar effects, reducing the ability of uPAR expressing cells to invade, and helping maintain the integrity of the cartilage.

In the healthy joint there is little uPAR expression; during arthritis, infiltrating leukocytes express both uPA and uPAR, and both chondrocytes and synoviocytes express uPAR in response to inflammatory cytokines (Busso et al. (1997) Ann Rheum Dis 56(9), 550-557; Guiducci et al. (2005) Clin Exp Rheumatol 23(3), 364-372; Schwab et al. (2001) Histochem Cell Biol 115(4), 317-323), and destructive neovascularization of the cartilage occurs as uPAR-expressing endothelial-cells invade. The potential effects of a uPAR antagonist have been demonstrated by systemic delivery of uPA-ATF to mice with collagen-induced arthritis, which reduced incidence and severity of disease, and extent of cartilage neovascularization (Apparailly et al. (2002) Gene Therapy 9(3), 192-200). In the absence of inflammation, CYTL1 is strongly expressed in articular cartilage, but its expression is dramatically reduced in response to inflammatory signals. This loss of uPA-antagonist activity may facilitate cellular invasion and extracellular matrix remodeling. Ongoing phenotypic analysis of CYTL1 gene deficient and transgenic mice should help to establish the in vivo role.

A role for uPAR in bone homeostasis has recently been discerned through study of uPAR-deficient mice (Furlan et al. (2007) J Bone Miner Res 22(9), 1387-1396). In the absence of uPAR expression, bone mineral density was elevated and bone volume was reduced. Enhanced osteoblast activity and a defect in adhesion of osteoclasts to bone surfaces may account for this phenotype. This presents a mechanism whereby CYTL1 expression in cartilage may affect matrix remodeling in adjacent bone.

Binding of CYTL1 to uPAR, as quantified by BIAcore analysis, is of intermediate affinity, approximately 1 μM. This is significantly lower than the typical range of affinity of cytokines for their cell-surface receptors. There may be an as yet unidentified co-receptor for CYTL1 which, in addition to presenting a mechanism for signal transduction, could increase the affinity of CYTL1 for uPAR at the cell surface, rendering it a more potent antagonist of the uPA-uPAR interaction. Even in the absence of a co-receptor, it is conceivable that CYTL1 would reach sufficiently high local concentrations to effectively compete with uPA. Careful analysis of the data from a large-scale gene expression study (Kumar et al. (2001) Osteoarthritis and Cartilage/OARS, Osteoarthritis Research Society 9(7), 641-653) reveals that CYTL1 is abundantly represented at the mRNA level, more so than any other cytokine. This also appears true at the protein level in a proteomic analysis of articular cartilage (Hermansson et al. (2004) J Biol Chem 279(42), 43514-43521), in which CYTL1 is readily detected in silver-stained 2-D gels. The interaction between CYTL1 and heparin presents a mechanism whereby the local concentration of CYTL1 may be boosted. While heparin is an unlikely physiological binding-partner, the abundant highly-sulfated glycosaminoglycans of cartilage may bind CYTL1, and localize it near the site of expression. Furthermore, the potentiation of binding of CYTL1 to uPAR achieved by heparin suggests that glycosaminoglycans either in the extracellular matrix or at the cell surface may enhance the affinity of CYTL1 for uPAR.

During preparation of this manuscript another study of CYTL1 was released (Kim et al. (2007) J Biol Chem In press. PMID: 17644814). The authors find that CYTL1 promotes differentiation of mesenchymal cells into chondrocytes. We have observed binding of CYTL1 AP to ATDC5 cells differentiating into chondrocytes (Supplemental FIG. 2a), and we have shown uPAR expression by these cells by qRT-PCR (Supplemental FIG. 2b).

Given that the molecular action of CYTL1 is so atypical for a member of the four-helical cytokine family, the question of whether CYTL1 truly is a member of this family remains open, and will not be answered without structural characterization. It is worth noting that many members of the family are pleiotropic, playing very different functional roles in different cellular compartments. Leptin, for example, acts both systemically as a regulator of lipid metabolism, and locally, regulating hematopoiesis (Wauters et al. (2000) Eur. J. Endocrin. 143(3), 293-311) and the development of regulatory T-cells (De Rosa et al. (2007) Immunity 26(2), 241-255). Similarly, it may be that CYTL1, expressed by CD34+ hematopoietic progenitor cells retains a more conventional cytokine-like function, but has adopted a very different function in cartilage and in the vascular wall.

TABLE 1

| | | |
|---|---|---|
| CYTL1 | FORWARD PRIMER | 5′CTG AGG ATT CCT GTG TGA GGT A 3′ |
| | PROBE | 5′CCC GGC TTT ACC TGG ACA TCC A 3′ |
| | REVERSE PRIMER | 5′TTG GCC AGC ACA CAG TAG TTA 3′ |
| β-actin | FORWARD PRIMER | 5′TCA TGA AGT GTG ACG TTG ACA T 3′ |
| | PROBE | 5′TGC ATC CTG TCA GCA ATG CCT G 3′ |
| | REVERSE PRIMER | 5′GGA GCA ATG ATC TTG ATC TTC A 3′ |
| uPAR | FORWARD PRIMER | 5′CCC TCC AGA GCA CAG AAA G 3′ |
| | PROBE | 5′CCT CGG GTG TAG TCC TCA TCC TTC A 3′ |
| | REVERSE PRIMER | 5′GGT TGC TAT GGA AAC CTG CT 3′ |
| GAPDH | FORWARD PRIMER | 5′CCG CAT CTT CTT GTG CAG 3′ |
| | PROBE | 5′TGC CAG CCT CGT CCC GTA GA 3′ |
| | REVERSE PRIMER | 5′ACA CCG ACC TTC ACC ATT TT 3′ |
| Aggrecan | FORWARD PRIMER | 5′CTG ACC CAC TGT CAA AGC AC 3′ |
| | PROBE | 5′CCT TCT GCT TCC GAG GTG TGT CAG T 3′ |
| | REVERSE PRIMER | 5′CAG AGG GTG ATG TGG GTG TA 3′ |
| ColLagen II | FORWARD PRIMER | 5′TGC GAT GAC ATT ATC TGT GAA G 3′ |
| | PROBE | 5′CCC GAG ATC CCC TTC GGA GAG T 3′ |
| | REVERSE PRIMER | 5′TGA TAT CTC CAG GTT CTC CTT TC 3′ |

1. A method of inhibiting the interaction of a urokinase-type plasminogen activator (uPA) and a urokinase-type plasminogen activator receptor (uPAR) comprising contacting a mixture comprising uPA and uPAR with a cytokine-like 1 (CYTL1) polypeptide or an agonist thereof.

2. The method of claim 1 wherein said contacting is performed in vitro.

3. The method of claim 1 wherein said contacting is performed in vivo.

4. The method of claim 1 wherein said mixture comprises cells expressing uPA and uPAR.

5. The method of claim 4 wherein said cells are cancer cells.

6. The method of claim 5 wherein said cancer is selected from the group consisting of carcinoma, lymphoma, blastoma, sarcoma, and leukemia.

7. The method of claim 5 wherein said cancer is selected from the group consisting of breast cancer, prostate cancer, colon cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma, kidney cancer, vulval cancer, thyroid cancer, hepatic carcinoma and various types of head and neck cancer.

8. The method of claim 1 wherein said agonist is selected from the group consisting of CYTL1 fragments, CYTL1 amino acid variants, agonist anti-CYTL1 antibodies and fragments thereof, peptides and non-peptide small molecules capable of binding uPAR.

9. A method of inhibiting a urokinase-type plasminogen activator (uPA) biological activity comprising contacting a cell expressing a urokinase-type plasminogen activator receptor (uPAR) and uPA in vivo with an effective amount of a CYTL1 or an agonist thereof.

10. The method of claim 9 wherein said cell is a cancer cell.

11. The method of claim 10 wherein said cancer is selected from the group consisting of carcinoma, lymphoma, blastoma, sarcoma, and leukemia.

12. The method of claim 10 wherein said cancer is selected from the group consisting of breast cancer, prostate cancer, colon cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma, kidney cancer, vulval cancer, thyroid cancer, hepatic carcinoma and various types of head and neck cancer.

13. The method of claim 9 wherein said cell is an endothelial cell.

14. The method of claim 9 wherein said agonist is selected from the group consisting of CYTL1 fragments, CYTL1 amino acid variants, agonist anti-CYTL1 antibodies and fragments thereof, peptides and non-peptide small molecules capable of binding uPAR.

15. A method for inhibiting tumor formation or tumor metastasis in a mammalian subject comprising administering to said subject an effective amount of CYTL1 or an agonist thereof.

16. The method of claim 15 wherein said mammalian subject is a human patient.

17. The method of claim 16 wherein said tumor is a cancer.

18. The method of claim 18 wherein said cancer is selected from the group consisting of carcinoma, lymphoma, blastoma, sarcoma, and leukemia.

19. The method of claim 18 wherein said cancer is selected from the group consisting of breast cancer, prostate cancer, colon cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma, kidney cancer, vulval cancer, thyroid cancer, hepatic carcinoma and various types of head and neck cancer.

20. The method of claim 16 wherein said agonist is selected from the group consisting of CYTL1 fragments, CYTL1 amino acid variants, agonist anti-CYTL1 antibodies and fragments thereof, peptides and non-peptide small molecules capable of binding uPAR.

21. The method of claim 20 wherein said CYTL1 variant has at least about 70% identity to the amino acid sequence of CYTL1 of SEQ ID NO: 2.

22. The method of claim 20 wherein said CYTL1 variant has at least about 80% identity to the amino acid sequence of CYTL1 of SEQ ID NO: 2.

23. The method of claim 20 wherein said CYTL1 variant has at least about 85% identity to the amino acid sequence of CYTL1 of SEQ ID NO: 2.

24. The method of claim 20 wherein said CYTL1 variant has at least about 90% identity to the amino acid sequence of CYTL1 of SEQ ID NO: 2.

25. The method of claim 20 wherein said CYTL1 variant has at least about 95% identity to the amino acid sequence of CYTL1 of SEQ ID NO: 2.

26. The method of claim 20 wherein said CYTL1 variant has at least about 99% identity to the amino acid sequence of CYTL1 of SEQ ID NO: 2.

27. The method of claim 20 wherein said CYTL1 variant is a naturally occurring variant of the CYTL1 polypeptide of SEQ ID NO: 2.

28. The method of claim 20 wherein said agonist is an anti-CYTL1 antibody or a fragment thereof.

29. The method of claim 28 wherein said antibody is a monoclonal antibody or a fragment thereof.

30. The method of claim 29 wherein said monoclonal antibody is chimeric, humanized or human.

31. The method of claim 29 wherein the antibody fragment is selected from the group consisting of Fab, Fab′, F(ab″)₂, and Fv fragments; diabodies; linear antibodies; single-chain antibody molecules; and multispecific antibodies formed from antibody fragment(s).

32. A method for inhibiting angiogenesis in a mammalian subject comprising administering to said subject an effective amount of CYTL1 or an agonist thereof.

33. The method of claim 32 wherein said mammalian subject is a human patient.

34. The method of claim 33 wherein said angiogenesis is tumor angiogenesis.

35. The method of claim 34 wherein said tumor is selected from the group consisting of carcinoma, lymphoma, blastoma, sarcoma, and leukemia.

36. The method of claim 34 wherein said tumor is selected from the group consisting of breast cancer, prostate cancer, colon cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma, kidney cancer, vulval cancer, thyroid cancer, hepatic carcinoma and various types of head and neck cancer.

37. The method of claim 33 wherein said agonist is selected from the group consisting of CYTL1 fragments, CYTL1 amino acid variants, agonist anti-CYTL1 antibodies and fragments thereof, peptides and non-peptide small molecules capable of binding uPAR.

38. The method of claim 37 wherein said CYTL1 variant has at least about 70% identity to the amino acid sequence of CYTL1 of SEQ ID NO: 2.

39. The method of claim 37 wherein said CYTL1 variant has at least about 80% identity to the amino acid sequence of CYTL1 of SEQ ID NO: 2.

40. The method of claim 37 wherein said CYTL1 variant has at least about 85% identity to the amino acid sequence of CYTL1 of SEQ ID NO: 2.

41. The method of claim 37 wherein said CYTL1 variant has at least about 90% identity to the amino acid sequence of CYTL1 of SEQ ID NO: 2.

42. The method of claim 37 wherein said CYTL1 variant has at least about 95% identity to the amino acid sequence of CYTL1 of SEQ ID NO: 2.

43. The method of claim 37 wherein said CYTL1 variant has at least about 99% identity to the amino acid sequence of CYTL1 of SEQ ID NO: 2.

44. The method of claim 37 wherein said CYTL1 variant is a naturally occurring variant of the CYTL1 polypeptide of SEQ ID NO: 2.

45. The method of claim 37 wherein said agonist is an anti-CYTL1 antibody or a fragment thereof.

46. The method of claim 45 wherein said antibody is a monoclonal antibody or a fragment thereof.

47. The method of claim 46 wherein said monoclonal antibody is chimeric, humanized or human.

48. The method of claim 46 wherein the antibody fragment is selected from the group consisting of Fab, Fab′, F(ab′)₂, and Fv fragments; diabodies; linear antibodies; single-chain antibody molecules; and multispecific antibodies formed from antibody fragment(s).

49. A method of screening for an antagonist of urokinase-type plasminogen activator (uPA), comprising: (a) incubating a mixture containing a urokinase-type plasminogen activator receptor (uPAR) and a CYTL1 or an agonist thereof with a candidate antagonist and (b) measuring the ability of said candidate antagonist to competitively inhibit the binding of said uPA or agonist thereof to said uPAR.

50. A method of reducing retinal neovascularization in a mammalian subject comprising administering to said subject an effective amount of CYTL1 or an agonist thereof.

52. A method of reducing the incidence or severity of arthritis in a mammalian subject comprising administering to said subject an effectice amount of CYTL1 or an agonist thereof.


SEQUENCE LISTING (44 sequences)

SEQ ID NO: 1 - DNA, 998 bp, Homo sapiens
  CDS (29)..(436)
gggcgaggctgcaccagcgcctggcaccatgaggacgcctgggcctctgcccgtgctgctgctgctcctggcgggagcccccgccgcgcggcccactcccccgacctgctactcccgcatgcgggccctgagccaggagatcacccgcgacttcaacctcctgcaggtctcggagccctcggagccatgtgtgagatacctgcccaggctgtacctggacatacacaattactgtgtgctggacaagctgcgggactttgtggcctcgcccccgtgttggaaagtggcccaggtagattccttgaaggacaaagcacggaagctgtacaccatcatgaactcgttctgcaggagagatttggtattcctgttggatgactgcaatgccttggaatacccaatcccagtgactacggtcctgccagatcgtcagcgctaagggaactgagaccagagaaagaacccaagagaactaaagttatgtcagctacccagacttaatgggccagagccatgaccctcacaggtcttgtgttagttgtatctgaaactgttatgtatctctctaccttctggaaaacagggctggtattcctacccaggaacctcctttgagcatagagttagcaaccatgcttctcattcccttgactcatgtcttgccaggatggttgatacacagcatgttgatttggtcactaaaaagaagaaaaggactaacaagcttcacttttatgaacaactattttgagaacatgcacaatagtatgtttttattactggtttaatggagtaatggtacttttattctttcttgatagaaacctgcttacatttaaccaagcttctattatgcctttttctaacacagactttcttcactgtctttcatttaaaaagaaattaatgctcttaagatatatattttacgtagtgctgacaggacccactctttcattgaaaggtgatgaaaatcaaataaagaatctcttcacatg

SEQ ID NO: 2 - protein, 136 aa, Homo sapiens
MRTPGPLPVLLLLLAGAPAARPTPPTCYSRMRALSQEITRDFNLLQVSEPSEPCVRYLPRLYLDIHNYCVLDKLRDFVASPPCWKVAQVDSLKDKARKLYTIMNSFCRRDLVFLLDDCNALEYPIPVTTVLPDRQR

SEQ ID NO: 3 - DNA, 21 bp, Artificial Sequence
  source: /note=“Description of Artificial Sequence Synthetic primer”
cccacctgctactctcggatg

SEQ ID NO: 4 - DNA, 23 bp, Artificial Sequence
  source: /note=“Description of Artificial Sequence Synthetic primer”
ggcaggtctaacagtggcactaa

SEQ ID NO: 5 - DNA, 18 bp, Artificial Sequence
  source: /note=“Description of Artificial Sequence Synthetic primer”
tcccccgacctgctactc

SEQ ID NO: 6 - DNA, 23 bp, Artificial Sequence
  source: /note=“Description of Artificial Sequence Synthetic primer”
cctgtgagggtcatggctctggc

SEQ ID NO: 7 - protein, 114 aa, Homo sapiens
TPPTCYSRMRALSQEITRDFNLLQVSEPSEPCVRYLPRLYLDIHNYCVLDKLRDFVASPPCWKVAQVDSLKDKARKLYTIMNSFCRRDLVFLLDDCNALEYPIPVTTVLPDRQR

SEQ ID NO: 8 - protein, 114 aa, Pan troglodytes
TPPTCYSRMRALSQEITRDFQLLQVSEPSEPCVRYLPRLYLDIHNYCVLDKLRDFVASPPCWKVAQVDSLKDKARKLYTIMNSFCRRDLVFLLDDCNALEYPIPVTTVLPDRQR

SEQ ID NO: 9 - protein, 114 aa, Macaca mulatta
TPPTCYSRMRALSQEITRDFQLLQSSEPSEPCVRYLPRLYLDIHNYCVLDKLRDFVASPQCWKAAQVDSLKDKARKLYTIMNSFCRRDLVFLLDDCNALEYPIPVTTVLPDHQR

SEQ ID NO: 10 - protein, 113 aa, Bos taurus
TPPTCYSRMLALSREITSDFQSLQATEPSEACVRYPRLYLDIHNYCVLAKLRDFVASPHCWKLAQVDALKDKVRKLYTIMNSFCRRDLVFLTDDCNALEYPVLVTTVLPDHQS

SEQ ID NO: 11 - protein, 114 aa, Mus musculus
APPTCYSRMLTLSREIMADFQSLQASEPEDSCVRYLPRLYLDIHNYCVLAKLRDFVASPQCWKMAEVDTLKDRVRKLYTIMNSFCRRDLVFLSDDCSALEDPIPEATGPPDWQS

SEQ ID NO: 12 - protein, 114 aa, Rattus norvegicus
APPTCYSRMLTLSREIMADFQSLQASEPEDSCVRYLPRLYLDIHNYCVLTKLRDFVASPQCWKMAEVDMLKDRVRKLYTIMNSFCRRDLVFLSDDCSVLEHPIPETTDPPDWQS

SEQ ID NO: 13 - protein, 121 aa, Monodelphis domestica
APPTCYSRMLNLSKEIMDSFNTLQNEEPLEPCVEGLPRLYLDIHNYCVLAKLRDFVASPRCRKVTQVGALKDKVRKLYTIMNSFCRRDLVFLSDDCEALENPIQAPTTVRSQVRSELKEVP

SEQ ID NO: 14 - protein, 115 aa, Gallus gallus
APPTCYSRVLSLSKEITESFKELQTSKAKDPCVETLPRLYLDIHNYCVLTKLRDFVAYPRCERVLEVSELKEKARSLYTIMISYCRRDLAFLTDDCNALESPIQSSIESSEDIDS

SEQ ID NO: 15 - protein, 114 aa, Rana sp.
APPTCYSRVFSLSKEITDSFQRIQKAAPPGPCLDALPTLYLDIHNSCVMTKLRTFISAPRCGRMPRVEALKKRVRNLYNIMNSICKRDLVFFTDDCEALEMPLSVFHPVTEPSR

SEQ ID NO: 16 - protein, 114 aa, Xenopus laevis
APPTCYSKVFTMSKEITDAFQRIQKTAPPGPCLDALPTLYLDIHNSCVMNKLRTFISAPRCGRMPRVEVLKRRVRKLYNIMNSVCKRDLVFFTDDCAALEVPLSTFRPITEPSR

SEQ ID NO: 17 - protein, 126 aa, Paralichthys olivaceus
APPTCYSRALGLSKEVMALLDKIHTYHRTKTCAEVLPTIFLDVHNSCVTTKLRDFLYVVLNHPNQYCRERPRMVLLKRKIQNLYTIITRICYRDLVFFTDDCEAIDTGHSSPYYAEDRLQLLQEER

SEQ ID NO: 18 - protein, 126 aa, Hippoglossus hippoglossus
APPTCYSRALGLSKEVMALLDKIHTYHRTKTCAEVLPTIFLDVHNACITTKLRDFLYVVLNHPNQYCRERPRMVLLKRKIQSLYTIITRMCYRDLVFFTDDCEAIDTGHTSPYYAEDRLQLLQEER

SEQ ID NO: 19 - protein, 125 aa, Fugu rubripes
APPTCYSRALGLSKEVMTLLDKIHTSERTKTCAEVLPTIFIDVHNSCVTSKLRDFLYVVLNHPDQSCRKRPRMMLLKRKVQNLYTIITRLCYRDLVFFMDDCEAIDTGHGTPHYTEDRLQLLEER

SEQ ID NO: 20 - protein, 126 aa, Oryzias latipes
APPTCYSRALELSREIMTLLDRVHTSHRTKTCAEVLPTIFLDVHNACITNKLRDFLYVVLNHPSQSCREKPRMKVLKGKIQNLYNIITRICYRDLVFFTDDCEAIDTGHSRPYVAEDRLQILQEER

SEQ ID NO: 21 - protein, 127 aa, Salmo salar
APPTCYSRVLGLSKEIMDLLEKLHNYHLTKTCVEILPKMFLDVHNSCIVTKLRDFLYVVENLPTRYCRERPRIVLLKRKVNNLYTIINRICYRDLVYFTDDCEAIETGQSTPRYGEDRLQLLVEEKR

SEQ ID NO: 22 - protein, 127 aa, Oncorhynchus mykiss
APPTCYSRVLGLSKEIIGLLGKLHNYHRTKTCVEILPKMFLDVQNSCINTKLRDFLYVMENLPTQYCRERPGIVLLKQKVTNLYRIINRICYRDLVYFTDDCEAIETGQSTPHYREDRLQMLVEEKR

SEQ ID NO: 23 - protein, 125 aa, Osmerus mordax
APLTCYSRVLGLSKEIMDLLDKVHNYHRTKTCVEILPKMFIDVHNSCLMPRLRDFLYVVENLPTQYCKERPRIVLLKRKVRNLYAIISKVCHRDLVYFTDDCEAIDTGHSVPRYGEDRLQLLEER

SEQ ID NO: 24 - protein, 121 aa, Pimephales promelas
SSRVLELSKEVMNTLDKIHKSIRTRTCAELLPKMFLDVHNSCIRSKLRDFLYVSENLPSESCREKPRIRLLKRRVQVLYSIINRACYRDMVFYTDDCEALETGNTSPRYTEDRLQQLIEDA

SEQ ID NO: 25 - protein, 126 aa, Danio rerio
APPTCYSRVLELSREITTILEKLHTTVRTKACAVLLPTVFLDPHNSCIRTKLRDLLYVVENLPVELCRERPRVRLLRRRTHTLLNIISRACHRDLVFFTDDCDAIDTGHSRPYVAEDRLQILQEER

SEQ ID NO: 26 - protein, 112 aa, Danio rerio
IPPTCYTKVLNMGRDITQRAAEIKQDYDTHRCTAHLPDLYIDVHNACVMSAMNTYLSMLSDLRERRCGYNREVKNLSAAIRQLYFIISQKCHGELVFTHDNCEALHRRPWRG

SEQ ID NO: 27 - DNA, 22 bp, Artificial Sequence
  source: /note=“Description of Artificial Sequence Synthetic primer”
ctgaggattcctgtgtgaggta

SEQ ID NO: 28 - DNA, 22 bp, Artificial Sequence
  source: /note=“Description of Artificial Sequence Synthetic probe”
cccggctttacctggacatcca

SEQ ID NO: 29 - DNA, 21 bp, Artificial Sequence
  source: /note=“Description of Artificial Sequence Synthetic primer”
ttggccagcacacagtagtta

SEQ ID NO: 30 - DNA, 22 bp, Artificial Sequence
  source: /note=“Description of Artificial Sequence Synthetic primer”
tcatgaagtgtgacgttgacat

SEQ ID NO: 31 - DNA, 22 bp, Artificial Sequence
  source: /note=“Description of Artificial Sequence Synthetic probe”
tgcatcctgtcagcaatgcctg

SEQ ID NO: 32 - DNA, 22 bp, Artificial Sequence
  source: /note=“Description of Artificial Sequence Synthetic primer”
ggagcaatgatcttgatcttca

SEQ ID NO: 33 - DNA, 19 bp, Artificial Sequence
  source: /note=“Description of Artificial Sequence Synthetic primer”
ccctccagagcacagaaag

SEQ ID NO: 34 - DNA, 25 bp, Artificial Sequence
  source: /note=“Description of Artificial Sequence Synthetic probe”
cctcgggtgtagtcctcatccttca

SEQ ID NO: 35 - DNA, 20 bp, Artificial Sequence
  source: /note=“Description of Artificial Sequence Synthetic primer”
ggttgctatggaaacctgct

SEQ ID NO: 36 - DNA, 18 bp, Artificial Sequence
  source: /note=“Description of Artificial Sequence Synthetic primer”
ccgcatcttcttgtgcag

SEQ ID NO: 37 - DNA, 20 bp, Artificial Sequence
  source: /note=“Description of Artificial Sequence Synthetic probe”
tgccagcctcgtcccgtaga

SEQ ID NO: 38 - DNA, 20 bp, Artificial Sequence
  source: /note=“Description of Artificial Sequence Synthetic primer”
acaccgaccttcaccatttt

SEQ ID NO: 39 - DNA, 20 bp, Artificial Sequence
  source: /note=“Description of Artificial Sequence Synthetic primer”
ctgacccactgtcaaagcac

SEQ ID NO: 40 - DNA, 25 bp, Artificial Sequence
  source: /note=“Description of Artificial Sequence Synthetic probe”
ccttctgcttccgaggtgtgtcagt

SEQ ID NO: 41 - DNA, 20 bp, Artificial Sequence
  source: /note=“Description of Artificial Sequence Synthetic primer”
cagagggtgatgtgggtgta

SEQ ID NO: 42 - DNA, 22 bp, Artificial Sequence
  source: /note=“Description of Artificial Sequence Synthetic primer”
tgcgatgacattatctgtgaag

SEQ ID NO: 43 - DNA, 22 bp, Artificial Sequence
  source: /note=“Description of Artificial Sequence Synthetic probe”
cccgagatccccttcggagagt

SEQ ID NO: 44 - DNA, 23 bp, Artificial Sequence
  source: /note=“Description of Artificial Sequence Synthetic primer”
tgatatctccaggttctcctttc